(19) United States
(12) Patent Application Publication
(10) Pub. No.: US 2024/0252583 A1
(43) Pub. Date: Aug. 1, 2024

(54) Compositions and Methods for Treating Persistent Postsurgical Pain

(71) Applicant: Creative Bio-Peptides, Inc., Potomac, MD (US)

(72) Inventor: Michael RUFF, Potomac, MD (US)

(21) Appl. No.: 18/560,768

(22) PCT Filed: Jun. 9, 2022
(86) PCT No.: PCT/US2022/032835

§ 371 Date: Nov. 14, 2023

Related U.S. Application Data
(60) Provisional application No. 63/208,517, filed on Jun. 9, 2021.

(51) Int. Cl. A61K 38/08; A61K 45/06; A61P 3/04; A61P 25/04

(52) U.S. Cl. CPC A61K 38/08; A61K 45/06; A61P 3/04; A61P 25/04

(57) ABSTRACT

Disclosed herein are polypeptides, derivatives thereof, and salts thereof, as well as pharmaceutical compositions containing these, useful alone or in combination with other therapies for treating weight loss, weight gain, or to maintain a healthy body weight in a subject. Also disclose herein are the use of the peptides disclosed herein for treating stress, depression, anxiety, and pain catastrophizing. In some cases, the subject can be suffering from persistent postsurgical pain.

### CROSS REFERENCE TO RELATED APPLICATIONS

This application claims the benefit of U.S. Provisional Application No. 63/208,517, filed Jun. 9, 2021 the disclosure of which is incorporated herein by reference in its entirety.

### STATEMENT AS TO FEDERALLY SPONSORED RESEARCH

This invention was made with government support under grant number MCDC-18-03-001 awarded by the Department of Defense.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY

Disclosed herein are methods of inducing weight gain in a subject in need thereof, inducing weight loss in a subject in need thereof, or inducing a healthy weight in a subject in need thereof. In some embodiments, the method can comprise: administering to the subject a therapeutically effective amount of a pharmaceutical composition to induce weight gain in the subject in need thereof, induce weight loss in the subject in need thereof, or to induce healthy weight in the subject in need thereof. In some embodiments, the pharmaceutical composition comprises: a polypeptide, a derivative thereof, or a salt thereof. In some embodiments, the polypeptide comprises at least five contiguous amino acids or derivatives thereof comprising the general formula: E-F-G-H-I, wherein: E can be D-Ser, D-Thr, D-Asn, D-Glu, D-Arg, D-Ile, or D-Leu, or a derivative of any of these; F can be D-Ser, D-Thr, D-Asp, or D-Asn, or a derivative of any of these; G can be D-Thr, D-Ser, D-Asn, D-Arg, D-Gln, D-Lys, or D-Trp, or a derivative of any of these; H can be D-Tyr, or a derivative thereof; and I can be D-Thr, D-Ser, D-Arg, or Gly, or a derivative of any of these. In some embodiments, the polypeptide, the derivative thereof, or the salt thereof comprises at least eight contiguous amino acids or derivatives thereof, comprising the general formula A-B-C-E-F-G-H-I. In some embodiments, A can be D-Ala, or a derivative thereof; B can be D-Ser, or D-Thr, or a derivative of any of these; C can be D-Ser, or D-Thr, or a derivative of any of these; E can be D-Ser, D-Thr, D-Asn, D-Glu, D-Arg, D-Ile, or D-Leu, or a derivative of any of these; F can be D-Ser, D-Thr, D-Asp, or D-Asn, or a derivative of any of these; G can be D-Thr, D-Ser, D-Asn, D-Arg, D-Gln, D-Lys, or D-Trp, or a derivative of any of these; H can be D-Tyr, or a derivative thereof; and I can be D-Thr, D-Ser, D-Arg, or Gly or a derivative of any of these. In some embodiments, the polypeptide or the salt thereof can be D-Thr, D-Thr, D-Asn, D-Tyr, and D-Thr or a salt thereof. In some embodiments, I can comprise a derivative of I. In some embodiments, I can be esterified, glycosylated, or amidated at the C terminus. In some embodiments, the pharmaceutical composition can be in unit dose form. In some embodiments, the pharmaceutical composition can further comprise a pharmaceutically acceptable: excipient, diluent, carrier, or a combination thereof. In some embodiments, the method can further comprise blocking neuropathic pain, enhancing opioid receptor analgesia, and promoting repair of neurons in the subject in need thereof. In some embodiments, the method can further comprise blocking ligand binding to a CX3CR1 receptor, reducing stress, or both in the subject in need thereof. In some embodiments, the subject has pain catastrophizing. In some embodiments, the method can further comprise increasing dopamine levels in the subject in need thereof. In some embodiments, the subject can have persistent postsurgical pain (PPSP). In some embodiments, the administering can at least partially block the neuropathic pain. In some embodiments, the administering can enhance opioid receptor analgesia. In some embodiments, the administering can result in the repair of neurons. In some embodiments, the administering can be daily, weekly, or monthly. In some embodiments, administering can be once, twice, three, or four times per day. In some embodiments, the pharmaceutical composition can be administered for about: one day, two days, three days, four days, five days, six days, one week, two weeks, three weeks, four weeks, five weeks, one month, two months, three months, four months, five months, six months, seven months, eight months, nine months, ten months, eleven months, one year, two years, or for life. In some embodiments, the pharmaceutical composition can comprise the polypeptide, the derivative thereof, or the salt thereof in an amount of from about 0.005 mg to about 1000 mg. In some embodiments, the pharmaceutical composition can be administered by: an oral route, an injection route, a sublingual route, a buccal route, a rectal route, a vaginal route, an ocular route, an otic route, a nasal route, an internasal route, an inhalation route, a cutaneous route, a subcutaneous route, an intramuscular route, an intravenous route, a systemic route, a local route, a transdermal route, or any combination thereof. In some embodiments, the pharmaceutical composition can be formulated for oral administration. In some embodiments, the pharmaceutical composition can be in a form of a pill or a liquid. In some embodiments, a second therapy can be administered concurrently or consecutively. In some embodiments, the second therapy can comprise an gabapentinoid, an opioid, a voltage-gated sodium channel inhibitor, an anti-nerve growth factor, an nonsteroidal anti-inflammatory drug, aspirin, a corticosteroid, acetaminophen, a muscle relaxant, an anti-anxiety drug, an antidepressant, a cox-2 inhibitor, a local anesthetic, an anticonvulsant, a cannabinoid, an NMDA receptor antagonist, an α2-adrenergic receptor agonist or any combination thereof. In some embodiments, the pharmaceutical composition can further comprise the second therapy. In some embodiments, the subject can be diagnosed with pain prior to the administration. In some embodiments, the diagnoses can comprise an in vitro test, a physical exam, an imaging diagnostic or a combination thereof. In some embodiments, the subject can be a mammal. In some embodiments, the mammal can be a human.

Also disclosed herein are methods of administering a polypeptide to treat Persistent Postsurgical Pain (PPSP) in a subject in need thereof. In some embodiments, the method can comprise administering to the subject a therapeutically effective amount of a pharmaceutical composition to treat PPSP. In some embodiments, the pharmaceutical composition can comprise: a polypeptide, a derivative thereof, or a salt thereof. In some embodiments, the polypeptide can comprise at least five contiguous amino acids or derivatives thereof comprising the general formula: E-F-G-H-I. In some embodiments: E can be D-Ser, D-Thr, D-Asn, D-Glu, D-Arg, D-Ile, or D-Leu, or a derivative of any of these; F can be D-Ser, D-Thr, D-Asp, or D-Asn, or a derivative of any of these; G can be D-Thr, D-Ser, D-Asn, D-Arg, D-Gln, D-Lys, or D-Trp, or a derivative of any of these; H can be D-Tyr, or a derivative thereof; and I can be D-Thr, D-Ser, D-Arg, or Gly, or a derivative of any of these. In some embodiments, the polypeptide, the derivative thereof, or the salt thereof can comprise at least eight contiguous amino acids or derivatives thereof, comprising the general formula A-B-C-E-F-G-H-I. In some embodiments, A can be D-Ala, or a derivative thereof; B can be D-Ser, or D-Thr, or a derivative of any of these; C can be D-Ser, or D-Thr, or a derivative of any of these; E can be D-Ser, D-Thr, D-Asn, D-Glu, D-Arg, D-Ile, or D-Leu, or a derivative of any of these; F can be D-Ser, D-Thr, D-Asp, or D-Asn, or a derivative of any of these; G can be D-Thr, D-Ser, D-Asn, D-Arg, D-Gln, D-Lys, or D-Trp, or a derivative of any of these; H can be D-Tyr, or a derivative thereof; and I can be D-Thr, D-Ser, D-Arg, or Gly or a derivative of any of these. In some embodiments, the polypeptide or the salt thereof can be D-Thr, D-Thr, D-Asn, D-Tyr, and D-Thr or a salt thereof. In some embodiments, I can comprise a derivative. In some embodiments, I can be esterified, glycosylated, or amidated at the C terminus. In some embodiments, the pharmaceutical composition can be in unit dose form. In some embodiments, the pharmaceutical composition can further comprise an pharmaceutically acceptable: excipient, diluent, carrier, or any combination thereof. In some embodiments, the pharmaceutical composition can be formulated for oral administration. In some embodiments, the pharmaceutical composition can be in a form of a pill or a liquid.

### BRIEF DESCRIPTION OF THE DRAWINGS

FIG. 1 shows the ordinal pain scale, which is based on the subjective ranking of pain by a patient or subject.

FIG. 2 shows graphs of the pharmacokinetic properties of RAP-103 (R103). FIG. 2A shows the pharmacokinetic properties of RAP-103 with rapid brain entry by oral and intravenous dosing in rats and guinea pigs. FIG. 2B shows the plasma levels of RAP-103 for hours in non-human primates (monkeys).

FIG. 3 shows an exemplary peptide manufacturing process for RAP-103.

FIG. 4 shows graphs of mechanical allodynia and cold allodynia in rats experiencing diabetic neuropathic pain after treatment with streptozotocin (STZ). The rats received oral administration of RAP-103 or a control treatment for 7 days. The graphs show reductions in mechanical allodynia and cold allodynia after treatment with RAP-103.

FIG. 5 shows graphs of the levels of mRNA for cytokines and chemokines tested after oral administration for 7 days of RAP-103 or a control vehicle (H20) in rats experiencing neuropathic pain. The rats had reductions in spinal cord mRNA encoding cytokines and chemokines associated with persistent post-surgical pain (PPSP) and other persistent pain conditions.

FIG. 6A shows a comparison of morphine alone at different doses with morphine plus RAP-103 on acute post-incisional pain. Percent reversal of mechanical allodynia was calculated using data from t=60 min. % reversal=[(60 min threshold−pre-dose threshold)/(baseline threshold−predose threshold)]×100. Data were analyzed by two-way ANOVA followed by Sidak's multiple comparison test, ** p<0.01.

FIG. 6B shows a co-administration of RAP-103 potentiates morphine antinociception in acute post-incisional pain. Dose-response curves for morphine alone and morphine in combination with a fixed dose (0.5 mg/kg) of the chemokine receptor antagonist, RAP-103 are shown. The percent reversal of mechanical allodynia was calculated using data from t=60 min. The ED50 of morphine alone is 3.19 mg/kg; the ED50 of morphine plus RAP-103 is 1.42 mg/kg. Data were analyzed using the Student's paired one-tailed t-test, p=0.06.

FIG. 7 shows the effect of RAP-103 on excitatory synapse numbers in cultured hippocampal neurons. Quantification of excitatory synapses by overlap of PSD95 and vGlut immunolabeling. Synapse numbers significantly declined in cultured neurons treated with Aβd/t but were protected by RAP-103 (10-10 M). * p=0.048; ** p=0.002.

FIG. 8 shows that RAP-310 was an antagonist of CX3CR1 mediated human monocyte chemotaxis at 0.01 pM (38% of control migration) and 1 pM (24% of control migration).

### DETAILED DESCRIPTION

### Overview

Often, when a person is suffering from persistent pain such a persistent postsurgical pain, the person develops stress, depression, and may have weight loss or weight gain due to suffering. Often, when a subject is suffering from persistent postsurgical pain blocking neuropathic pain, enhancing opioid receptor analgesia, and promoting the repair of neurons can provide treatment for the subject. In some cases, peptides herein can block neuropathic pain by inhibiting inflammatory response in the peripheral and/or the central nervous system. In some cases, the peptides herein can enhance opioid receptor analgesia, for example peptides herein can promote greater analgesia of an opioid when the opioid is used in the treatment of a pain such as in the treatment of PPSP. In some cases, the peptides herein can promote repair of neurons. For example, the peptides herein can promote synapse and dendritic spine structure, function, formation, regeneration, or any combination thereof. In some cases, the regeneration of neurons can reduce pain in PPSP. In some cases, the regeneration of neurons can reduce stress.

More than 45 million surgical procedures are performed in the United States alone each year. It has been estimated that acute postoperative pain will develop into persistent postsurgical pain (PPSP) in 10% to 50% of individuals after common operations. Since chronic pain can be severe in up to 10% of these patients, PPSP represents a major clinical problem affecting at least 450,000 people each year.

Persistent postsurgical pain is defined as a clinical discomfort that lasts more than 3 months post-surgery without other causes of pain such as chronic infection or pain from a chronic condition preceding the surgery. According to the International Classification of Diseases, persistent postsurgical pain has greater intensity or different pain characteristics than preoperative pain and is a continuum of acute postsurgical pain that may develop after an asymptomatic period.

According to the International Classification of Diseases PPSP has different pain characteristics than pre-operative pain and can be a continuum of acute post-operative pain that may develop after an asymptomatic period.

Results from thoracic and other surgical procedures suggest multiple pathogenic mechanisms that include pre-, intra-, and postoperative factors. The International Association for the Study of Pain (IASP) definition of pain after thoracotomy can include “Pain that occurs or persists along a thoracotomy scar at least 2 months following surgical procedure.”

This persistent pain after surgery can be a very specific entity that may not be an inflammatory response alone, or from isolated nerve injuries but can be a combination of both which relate to mechanisms of peripheral and central sensitization. Nerve injury may be a prerequisite for the development of post-surgical chronic pain but may not be sufficient. Only a proportion of patients with intraoperative nerve damage may develop chronic pain and less than 50% of post-surgical chronic pain can show a neuropathic component. Further distinguishing PPSP from other chronic pain states such as neuropathic pain can be the failure of typical approved treatments for neuropathic pain, such as pregabalin/gabapentinoids, or anti-inflammatory agents like NSAIDS or steroids to provide meaningful or significant or any pain relief in this patient population. The pathophysiologic mechanisms of neuropathic pain and PPSP shares some features but can diverge in as yet not completely understood critical aspects, as can the treatments. PPSP remains a large unmet medical need.

In addition to the direct injury aspects of nerve damage from surgery that may lead to chronic pain, several studies identify a role of opioid receptors in the exaggeration of postoperative pain. For example, morphine treatment given before incision dramatically prolongs subsequent pain hypersensitivity produced by hind paw incision in animals. Fentanyl also produces immediate analgesia but exaggerates postoperative pain hypersensitivity. The enhancement of pain hypersensitivity observed in morphine-treated animals has been related to an increase in p38 MAP kinase activation in the dorsal spinal cord. Other evidence indicates that nerve injury downregulates mu opioid receptors and decreases opioid efficacy at the spinal level. These findings suggest that altered opioid pathways after surgeries facilitate the development of persistent postsurgical pain. Paradoxically when high opioid doses are used initially to treat acute pain, chronic pain may ensue. More recently a connection between opioid and chemokine receptors in pain has been revealed as it is now appreciated that activated chemokine receptors, which arise after nerve damage from surgeries, desensitize opioid receptors to promote pain by a process of receptor cross-desensitization. RAP-103, by blocking activation of select chemokine receptors, can enhance the analgesic effect of opioids for better pain treatment.

Axonal transection from surgery is the primary injury which leads to pain sensitization. When an axon is cut its distal end may degenerate and may be engulfed by inflammatory cells which can also sensitize individuals to chronic pain. Secondary processes involving innate immune responses to surgical injury seem to establish the chronic pain state. Even if postoperative pain resolves in most of these patients, surgery may sensitize patients to subsequent nociceptive stimuli which contributes to PPSP.

A mechanism that may support pain sensitization after surgery leading to persistent postsurgical pain include nociceptive inputs due to surgery produce local molecular changes such as nerve growth factor (NGF) and cytokine release and in primary sensory neurons of the dorsal root ganglia (DRG) including increased expression of acid-sensing ion channels 3 (ASICS3), transient receptor potential cation channel subfamily V member 1 (TRPV1), and mechanistic target of rapamycin (mTOR). The latter may control vesicular glutamate transporter 2 (VGLUT2) expression that generates an increased glutamatergic activity in the spinal cord. These changes may be responsible for peripheral pain sensitization that then influences spinal neuronal activity referred to as central pain sensitization. Central sensitization may depend upon increased expression of the α-amino-3-hydroxy-5-methyl-4-isoxazolepropionic acid receptor (AMPAR) and brain-derived neurotrophic factor (BDNF) release. Activation of AMPAR may account for extracellular signal-regulated kinase 1/2 activation (P-ERK1/2) leading to the development of sustained pain hypersensitivity. MAPK kinase p38 activation (P-p38) and chemokine ligand 2 (CCL2) may also contribute to surgery-induced central pain sensitization.

Different neurochemical changes may occur in primary afferent neurons and in the spinal cord, a site of central pain activation, in different persistent pain states. PPSP creates a unique pain state that is thought to involve sensitization of the nervous system and patients with this type of pain may be difficult to treat. The medical procedures such as those arising from surgery can damage peripheral neurons making them both generators of pain and while at the same time, non-responsive to or less responsive to traditional pain therapies, such as opioids and gabapentin. Because PPSP is refractory to conventional pain treatments, it was originally thought that the peptides herein would be unlikely to treat PPSP for all the reasons described above. However, recent work showing multiple actions of the peptides herein acting on multiple pathways at the same time suggests otherwise.

In some instances, the peptides herein can treat PPSP arising from medical procedures such as a thoracotomy, a cesarean section, a cardiac surgery, a breast surgery, an amputation, a total knee arthroplasty, a hip arthroplasty, a hernia repair, a cholecystectomy, a vasectomy, a cancer resection, a dental surgery, or any surgery.

In some instances, the peptides herein can reduce pain at a dose 5-fold to about 40-fold lower (weight-to-weight) than a commonly administered pain medication (e.g., an opioid or another conventional therapy). In some instances, the peptides herein can reduce pain at a dose 5-fold to about 35-fold lower (weight-to-weight) than a commonly administered pain medication. In some instances, the peptides herein can reduce pain at a dose 5-fold to about 30-fold lower (weight-to-weight) than a commonly administered pain medication. In some instances, the peptides herein can reduce pain at a dose 5-fold to about 25-fold lower (weight-to-weight) than a commonly administered pain medication. In some instances, the peptides herein can reduce pain at a dose 5-fold to about 20-fold lower (weight-to-weight) than a commonly administered pain medication. In some instances, the peptides herein can reduce pain at a dose 5-fold to about 15-fold lower (weight-to-weight) than a commonly administered pain medication. In some instances, the peptides herein can reduce pain at a dose 5-fold to about 10-fold lower (weight-to-weight) than a commonly administered pain medication. In some instances, the peptides herein can reduce pain at a dose of about: 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold lower (weight-to-weight) than a commonly administered pain medication.

In certain instances, the conventional therapy is an opioid. In certain instances, the opioid is hydrocodone, oxycodone, hydromorphone, meperidine oxymorphone, morphine, codeine, opium, pentazocine, tapentadol, tramadol, heroin or fentanyl. In certain instances, the opioid morphine. In some cases, the peptides herein can be administered concurrently or consecutively with a conventional therapy such as an opioid.

In certain instances, the peptides herein can reduce pain by about: 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold on a weight-to-weight basis as compared to a conventional pain management therapy typically used to treat PPSP. In some instances, a peptide herein may comprise up to eight or more than eight contiguous amino acids or derivatives thereof, comprising the general formula A-B-C-E-F-G-H-I, and wherein: A is D-Ala, or a derivative thereof; B is D-Ser, or D-Thr, or a derivative of any of these; C is D-Ser, or D-Thr, or a derivative of any of these; E is D-Ser, D-Thr, D-Asn, D-Glu, D-Arg, D-Ile, or D-Leu, or a derivative of any of these; F is D-Ser, D-Thr, D-Asp, or D-Asn, or a derivative of any of these; G is D-Thr, D-Ser, D-Asn, D-Arg, D-Gln, D-Lys, or D-Trp, or a derivative of any of these; H is D-Tyr, or a derivative thereof, and I is D-Thr, D-Ser, D-Arg, or Gly or a derivative of any of these. In some cases, a peptide herein can comprise at least five contiguous amino acids or derivatives thereof comprising the general formula: E-F-G-H-I, wherein: E is D-Ser, D-Thr, D-Asn, D-Glu, D-Arg, D-Ile, or D-Leu, or a derivative of any of these; wherein F is D-Ser, D-Thr, D-Asp, or D-Asn, or a derivative of any of these; wherein G is D-Thr, D-Ser, D-Asn, D-Arg, D-Gln, D-Lys, or D-Trp, or a derivative of any of these; wherein H is D-Tyr, or a derivative thereof; and wherein I is D-Thr, D-Ser, D-Arg, or Gly, or a derivative of any of these. In some cases, a conventional pain management therapy can comprise a gabapentinoid, an opioid, a voltage-gated sodium channel inhibitor, an anti-nerve growth factor, an nonsteroidal anti-inflammatory drug, aspirin, a corticosteroid, acetaminophen, a muscle relaxant, an anti-anxiety drug, an antidepressant, a cox-2 inhibitor, a local anesthetic, an anticonvulsant, a cannabinoid, an NMDA receptor antagonist, an α2-adrenergic receptor agonist or any combination thereof.

In some instances, peptides in the compositions and pharmaceutical formulations provided herein decreases pain by 1, 2, 3, 4, 5, 6, 7, 8, 9, or 10 points on the ordinal pain scale after administration of the peptide. In some cases, a peptide herein eliminates pain.

The multi-chemokine receptor antagonist peptide RAP-103 (All D-peptide-Thr-Thr-Asn-Tyr-Thr) is being developed to provide a non-opioid, potentially disease modifying treatment for PPSP. Previous work has shown proof-of-concept (POC) animal studies in neuropathic pain by partial nerve ligation and diabetes. In some cases, RAP-103 can be used to treat PPSP. A neuroprotective mechanism for RAP-103 pain effects may be established. Additional chronic pain patients that may benefit from RAP-103 (All-D-TTNYT) include those with spinal cord injury, chronic low-back pain, and post-herpetic neuralgia. Chemokines, molecules of the innate immune system that mediate inflammation, acting through receptors such as CCR2, CCR5, and CCR8 can promote pain by multiple mechanisms that cause sustained excitability of primary nociceptive neurons, desensitize endogenous opioid anti-pain effects, activate microglia and astrocytes, and cause peripheral monocyte infiltration into CNS. Blocking multiple chemokine receptors that can establish and sustain chronic pain with the multi-chemokine receptor antagonist (CRA) RAP-103 may be a non-opioid approach to pain treatment. Independent research by others shows the value of CRA's in diverse chronic pain conditions. RAP-103 is a CRA because of its ease of dosing, rapid entry into the CNS, lack of toxicity and side effects, and potential to treat the defining pathology of PPSP, axonal degeneration. Unlike current FDA approved chemokine antagonists (plerixafor, Maraviroc) which have significant safety concerns (allergic risks, need to be injected, “black-box” warning for hepatotoxicity), RAP-103 can be safe and may target multiple chemokine receptors (CCR2/CCR5/CCR8) implicated in pain states. A scale-up peptide manufacture may be conducted and may complete IND-enabling pre-clinical safety and PK/PD studies.

CX3CR1 is a well-known receptor involved in immune cell recruitment and inflammation. Pathological inflammation leads to pain stimulation and hence nociception. CX3CR1 and its ligands, particularly CX3CL1, modulate nociception via actions in the dorsal root ganglia and dorsal horn of the spinal cord. Blocking CX3CR1 responses with the peptides of the invention, such as RAP-103 (all-D-TTNYT) and RAP-310 (all-D-ASTTTNYT), will reduce pain is PPSP.

In some instances, the peptides provided herein are able to block CX3CR1. Typically, Fractalkine (CX3CL1) binding to microglial CX3CR1 induces the activation of several downstream signaling pathways, especially the activation of intracellular p38 MAPK pathway that leads to the release of CatS and IL-1β. The activation of this pathway is linked to nociceptive facilitation after nerve injury. Pain development in this model correlates with an increased dorsal horn microgliosis and increased expression of p38 in microglia. This chemokine pair CX3CL1/CX3CR1 is involved in persistent pain development and maintenance via neuron-microglia interaction in the dorsal horn, and upregulation of CX3CR1 expression is observed when microgliosis is present.

Anxiety, stress, depression and pain catastrophizing, from injury or emotional state, can be associated with increased post-operative pain. All of these states may enhance pain and mediate the transition from acute pain after surgery to chronic pain after surgery. Balancing innate immune responses with the peptides of the invention, such as RAP-103 and RAP-310, will reduce the nociceptive effects of anxiety, stress, depression, ideation of suicide, for example in a patient experiencing persistent pain such as PPSP.

In some instance, the peptides in the compositions and pharmaceutical formulations provided herein are able to reduce a stress, a depression, an anxiety, a pain catastrophizing, or any combination thereof. Neuropathic pain is a complex chronic condition characterized by various sensory, cognitive, and affective symptoms. A large percentage of patients with neuropathic pain are also afflicted with depression and anxiety disorders, a pattern that is also seen in animal models. Chronic pain may correspond with adaptations in several brain networks involved in mood, motivation, and reward. Chronic stress is also a major risk factor for depression. Stress modulates pain perception, resulting in either stress-induced analgesia or stress-induced hyperalgesia, as reported in both animal and human studies. The responses to stress include neural, endocrine, and behavioral changes, and built-in coping strategies are in place to address stressors. In some cases, the peptides herein can be used to treat pain catastrophizing. In some cases, pain catastrophizing can be characterized by the tendency to magnify the threat value of the pain stimulus, to feel helpless in the context of pain, or any combination thereof. In some cases, pain catastrophizing can comprise a relative inability to inhibit pain-related thoughts in anticipation of, during or following a painful event. Pain catastrophizing has been shown in many clinical and experimental studies as an important correlate of pain, pain-related disability, and outcome (e.g., after surgery and chronification), as such, the peptides herein (e.g. RAP-103) can be used to treat pain catastrophizing.

In some instances, the reduction of stress by way of administration of the peptides in the compositions and pharmaceutical formulations provided herein can be measured by use of the Perceived Stress Scale (PSS). The PSS is a widely used psychological instrument for measuring the perception of stress. It is a measure of the degree to which situations in a subject's life are appraised as stressful. Items were designed to tap how unpredictable, uncontrollable, and overloaded respondents find their lives. The scale also includes a number of direct queries about current levels of experienced stress. The PSS was designed for use in community samples with at least a junior high school education. The items are easy to understand, and the response alternatives are simple to grasp. Moreover, the questions are of a general nature and hence are relatively free of content specific to any subpopulation group. The questions in the PSS ask about feelings and thoughts during the last month. In each case, respondents are asked how often they felt a certain way. In some cases, a peptide herein can decrease stress of the PSS scale.

In some instances, the peptides herein can be used to increase dopamine levels in a subject. For example, the peptides disclosed herein can be used to increase dopamine levels in a subject by about: 5%, 10%, 15%, 20%, 25%, 30%, 35%, 40%, 45%, 50%, 55%, 60%, 65%, 70%, 75%, 80%, 85%, 90%, 95%, 99%, or 100% as compared to the dopamine levels in the subject before the administration of the peptides herein.

In some instances, the peptides in the compositions and pharmaceutical formulations provided herein can be used to decrease, increase, or maintain a subject's weight. For example, a peptide herein can be administered to a subject who is overweight to reduce the subject's weight. In some cases, peptides herein can be used to treat obesity or overweight. In some cases, peptides herein can be used to treat obesity class 1, obesity class 2, or obesity class 3. In some cases, overweight can comprise a BMI (Body mass index) of 25.0 to 29.9. In some cases, class 1 obesity can comprise a BMI of 30 to 34.9. In some cases, class 2 obesity can comprise a BMI of 35.0 to 39.9. In some cases, class 3 obesity can comprise a BMI greater than 40. In some cases, peptides herein can be used to reduce the BMI of a subject. In some cases, a peptide herein can be used by a subject to gain weight. For example, the peptides herein can be used to treat anorexia or other eating disorders. In some cases, the peptides herein can be used to maintain a body weight.

In some instances, the peptides in the compositions and pharmaceutical formulations provided herein are able to repair nerve damage such as that caused by or associated with PPSP. Repairing nerve damage by the peptides disclosed herein is described in U.S. Pub. No. 20210244788A1, which is incorporated herein by reference in its entirety.

In some instances, the peptides in the compositions and pharmaceutical formulations provided herein are able to promote weight gain by decreasing pain suffering and increasing appetite. Similarly, in certain instances, depression, stress, and the like due to suffering from pain can result in an increase in weight. Use of the peptides in the compositions and pharmaceutical formulations provided herein are able alleviate suffering, thereby increasing body weight of a subject in need thereof, decreasing body weight of a subject in need thereof, or maintaining a healthy body weight. Generally, a healthy body weight is having a body mass index (BMI) of between about 18.5 and 24.9. BMI is generally calculated by kg/m², where kg is a person's weight in kilograms and m² is their height in meters squared.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various aspects may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term persistent postsurgical pain (PPSP) is defined as a clinical discomfort that lasts more than 3 months post-surgery without other causes of pain such as chronic infection or pain from a chronic condition preceding the surgery.

As used herein, the term ‘about’ a number can refer to that number plus or minus 10% of that number. The term ‘about’ a range can refer to that range minus 10% of its lowest value and plus 10% of its greatest value.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement and include determining if an element may be present or not (for example, detection). These terms may include quantitative, qualitative or quantitative, and qualitative determinations. Assessing may be alternatively relative or absolute. “Detecting the presence of” includes determining the amount of something present, as well as determining whether it may be present or absent.

The terms “subject,” “individual,” or “patient” are often used interchangeably herein. A “subject” may be a biological entity containing expressed genetic materials. The biological entity may be a plant, animal, or microorganism, including, for example, bacteria, viruses, fungi, and protozoa. The subject may be tissues, cells and their progeny of a biological entity obtained in vivo or cultured in vitro. The subject may be a mammal. The mammal may be a human. The subject may be diagnosed or suspected of being at high risk for a disease. In some cases, the subject may not be necessarily diagnosed or suspected of being at high risk for the disease.

The term “at least partially” may refer to a qualitative condition that exhibits a partial range or degree of a feature or characteristic of interest. For example, at least partially may comprise a reduction in PPSP that is at least about: 5%, 10%, 20%, 30%, 40%, 50%, 60%, 70%, 75%, 80%, 85%, 90%, 95%, 99%, or 100% reduced relative to untreated.

The term “in vivo” may be used to describe an event that takes place in a subject's body.

The term “ex vivo” may be used to describe an event that takes place outside of a subject's body. An “ex vivo” assay may not be performed on a subject. Rather, it may be performed upon a sample separate from a subject. An example of an “ex vivo” assay performed on a sample may be an “in vitro” assay.

The term “in vitro” may be used to describe an event that takes place contained in a container for holding laboratory reagent such that it may be separated from the living biological source organism from which the material may be obtained. In vitro assays may encompass cell-based assays in which cells alive or dead are employed. In vitro assays may also encompass a cell-free assay in which no intact cells are employed.

As used herein, the terms “treatment” or “treating” are used in reference to a pharmaceutical or other intervention regimen for obtaining beneficial or desired results in the recipient such as preventing symptoms of PPSP from occurring or reducing or eliminating a pain, an inflammation, nerve damage or a combination thereof. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit may be achieved with the eradication or amelioration of one or more of the physiological symptoms associated with the underlying disorder such that an improvement may be observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, a “dose” can refer to a measured quantity of a therapeutic agent to be taken at one time.

As used herein, the term “unit dose” or “dosage form” may be used interchangeably and may be meant to refer to pharmaceutical drug products in the form in which they are marketed for use, with a specific mixture of active ingredients and inactive components or excipients, in a particular configuration, and apportioned into a particular dose to be delivered. The term “unit dose” may also sometimes encompass non-reusable packaging, although the FDA distinguishes between unit dose “packaging” or “dispensing”. More than one unit dose may refer to distinct pharmaceutical drug products packaged together, or to a single pharmaceutical drug product containing multiple drugs and/or doses. Types of unit doses may vary with the route of administration for drug delivery, and the substance(s) being delivered. A solid unit dose may be the solid form of a dose of a chemical compound used as a pharmaceutically acceptable drug or medication intended for administration or consumption.

As used herein, “pharmaceutically acceptable salt” may refer to pharmaceutical drug molecules, which may be formed as a weak acid or base, chemically made into their salt forms, most frequently as the hydrochloride, sodium, or sulfate salts. Drug products synthesized as salts may enhance drug dissolution, boost absorption into the bloodstream, facilitate therapeutic effects, and increase its effectiveness. Pharmaceutically acceptable salts may also facilitate the development of controlled-release dosage forms, improve drug stability, extend shelf life, enhance targeted drug delivery, and improve drug effectiveness.

The phrase “pharmaceutically acceptable excipient” as used herein may refer to a pharmaceutically acceptable material, composition or vehicle, such as a liquid or solid filler, diluent, excipient, carrier, solvent or encapsulating material.

As used herein, a “pharmaceutical agent” may refer to an agent or a therapy that may be used to prevent, diagnose, treat, or cure a disease, or combinations thereof. In some cases, a pharmaceutical agent can comprise a peptide disclosed herein or, in some aspects, a method described herein may comprise administering a therapeutically effective amount of these to a subject. In some cases, a subject can be a mammal, for example a human.

As used herein, “agent” or “biologically active agent” may refer to a biological, pharmaceutical, or chemical compound or a salt of any of these. Non-limiting examples may include a simple or complex organic or inorganic molecule, a peptide, a protein, a nucleotide such as an engineered single stranded RNA, an engineered single stranded DNA, an alternative nucleic acid, a protein, a carbohydrate, a toxin, or a chemotherapeutic compound. Various compounds may be synthesized, for example, small molecules and oligomers (e.g., oligopeptides and oligonucleotides), or synthetic organic compounds based on various core structures. In addition, various natural sources may provide compounds for screening, such as plant or animal extracts, and the like.

As used herein, the terms “effective amount” or “therapeutically effective amount” of a drug used to treat a disease may be an amount that may reduce the severity of a disease, reduce the severity of one or more symptoms associated with the disease or its treatment, or delay the onset of more serious symptoms or a more serious disease that may occur with some frequency following the treated condition. An “effective amount” may be determined empirically and in a routine manner, in relation to the stated purpose.

As used herein, “time to peak plasma concentration” can refer to the time required for a drug to reach peak concentration in plasma. Peak concentration in plasma can be defined as the plasma concentration that a drug achieves in a specified compartment or test area of the body after the drug has been administered and before the administration of a second dose.

The term “substantially” or “essentially” can refer to a qualitative condition that exhibits an entire or nearly total range or degree of a feature or characteristic of interest. In some cases, substantially can refer to a pain level that varies from a mean or median pain level by about plus or minus: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. For example, substantially can refer to: 70%, 75%, 80%, 85%, 90%, 95%, 99%, or 100% reduced pain. In some cases, substantially can refer to at least about 70%, 75%, 80%, 85%, 90%, 95%, 99%, or 100% of the total range or degree of a feature or characteristic of interest.

As used herein, “HPLC” can refer to high-performance liquid chromatography (formerly referred to as high-pressure liquid chromatography), which is a technique in analytical chemistry used to separate, identify, and quantify each component in a mixture. HPLC can be a common technique used in pharmaceutical development, as it can be a method to ensure product purity. The terms peptide and polypeptide can be used interchangeably herein.

The term “fragment,” as used herein, may be a portion of a sequence, a subset that may be shorter than a full-length sequence. A fragment may be a portion of a gene. A fragment may be a portion of a peptide or protein. A fragment may be a portion of an amino acid sequence. A fragment may be a portion of an oligonucleotide sequence. A fragment may be less than about: 20, 30, 40, 50 amino acids in length. A fragment may be about 10%, about 15%, about 20%, about 25%, about 30%, about 35%, about 40%, about 50%, about 60% or about 70% of the total length of an amino acid sequence or a nucleotide sequence. A fragment may be less than about: 20, 30, 40, 50 oligonucleotides in length.

As used herein, amino acids can be referenced by their one or three letter codes, which are shown in Table 1.

TABLE 1
Amino acids

| | | |
|---|---|---|
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Amino acids, depending upon the configuration at the alpha carbon, can be D or L-excepting glycine which does not contain four non-identical substituents on its alpha carbon atom. The designations D and L should not be confused with one letter amino acid codes. In some embodiments, D amino acids are designated with a D in front of the amino acid (e.g., D-Ser, dA). If the amino acid has four non-identical substituents on its alpha carbon atom, and the amino acid is not designated with a D in front of the amino acid, the amino acid can be of the L configuration. The amino acid glycine, lacking four non-identical substituents on its alpha carbon atom, may not be D or L.

The section headings used herein are for organizational purposes only and are not to be construed as limiting the subject matter described.

Peptides herein can include peptides in Table 2.

TABLE 2
Peptide sequences
Peptide Sequences

TTNYT (RAP-103)
SSTYR
STNYT
NTSYG
dASTTTNYT-NH2 (DAPTA)
ASTTTNYT (RAP-310)

Wherein in the table, peptides TTNYT, SSTYR, STNYT and ASTTTNYT are all D-peptides such that each amino acid in the peptide is in the D configuration. Wherein in the table, each amino acid of peptide NTSYG, except for glycine, is in the D configuration. And wherein in the table, dASTTTNYT-NH2 the alanine is in the D configuration, and all other amino acids in peptide 5 are in the L configuration.

### Peptide Compositions

In some instances, a composition is provided which comprises a polypeptide wherein the polypeptide comprises at least five contiguous amino acids or derivatives thereof comprising the general formula: E-F-G-H-I, wherein: E is D-Ser, D-Thr, D-Asn, D-Glu, D-Arg, D-Ile, or D-Leu, or a derivative of any of these; wherein F is D-Ser, D-Thr, D-Asp, or D-Asn, or a derivative of any of these; wherein G is D-Thr, D-Ser, D-Asn, D-Arg, D-Gln, D-Lys, or D-Trp, or a derivative of any of these; wherein H is D-Tyr, or a derivative thereof; and wherein I is D-Thr, D-Ser, D-Arg, or Gly, or a derivative of any of these.

In some instances, E can be D-Thr. In some instances, F can be D-Thr. In some instances, E can be D-Thr and F can be D-Thr. In some instances, G can be D-Asn. In some instances, F can be D-Thr and G can be D-Asn. In some instances, E can be D-Thr, F can be D-Thr, and G can be D-Asn. In some instances, H can be D-Tyr. In some instances, E can be D-Thr and H can be D-Tyr. In some instances, G can be D-Asn and H can be D-Tyr. In some instances, I can be D-Thr. In some instances, E can be D-Thr, F can be D-Thr, G can be D-Asn, H can be D-Tyr, and I can be D-Thr.

In some instances, the polypeptide may comprise at least eight contiguous amino acids or derivatives thereof, comprising the general formula A-B-C-E-F-G-H-I, and wherein: A is D-Ala, or a derivative thereof; B is D-Ser, or D-Thr, or a derivative of any of these; C is D-Ser, or D-Thr, or a derivative of any of these; E is D-Ser, D-Thr, D-Asn, D-Glu, D-Arg, D-Ile, or D-Leu, or a derivative of any of these; F is D-Ser, D-Thr, D-Asp, or D-Asn, or a derivative of any of these; G is D-Thr, D-Ser, D-Asn, D-Arg, D-Gln, D-Lys, or D-Trp, or a derivative of any of these; H is D-Tyr, or a derivative thereof, and I is D-Thr, D-Ser, D-Arg, or a derivative of any of these.

In some cases, A can be D-Ala. In some instances, B can be D-Ser. In some instances, B can be D-Thr. In some instances, A can be D-Ala and B can be D-Ser. In some instances, A can be D-Ala and B can be D-Thr. In some instances, C can be D-Ser. In some instances, C can be D-Thr. In some instances, B can be D-Ser and C can be D-Ser. In some instances, B can be D-Thr and C can be D-Ser. In some instances, B can be D-Thr and C can be D-Thr. In some instances, E can be D-Thr. In some instances, F can be D-Thr. In some instances, E can be D-Thr and F can be D-Thr. In some instances, G can be D-Asn. In some instances, F can be D-Thr and G can be D-Asn. In some instances, E can be D-Thr, F can be D-Thr, and G can be D-Asn. In some instances, H can be D-Tyr. In some instances, E can be D-Thr and H can be D-Tyr. In some instances, G can be D-Asn and H can be D-Tyr. In some instances, I can be D-Thr. In some instances, E can be D-Thr, F can be D-Thr, G can be D-Asn, H can be D-Tyr, and I can be D-Thr.

In some instances, the polypeptide sequence can comprise a sequence of ASTTTNYT, where each amino acid, individually, can be of the L or of the D configuration; in some instances, all amino acids in the sequence ASTTTNYT can be in the L configuration; in some instances, all amino acids int the sequence ASTTTNYT can be of the D configuration.

In some instances, the polypeptide sequence can comprise a sequence of TTNYT, where each amino acid, individually, can be of the L or of the D configuration; in some instances, all amino acids in the sequence TTNYT can be in the L configuration; in some instances, all amino acids in the sequence TTNTY can be of the D configuration. ASTTTNYT-NH2, where each amino acid, individually, can be of the L or of the D configuration; in some instances, all amino acids in the sequence ASTTTNYT-NH₂ can be in the L configuration; in some instances, all amino acids int the sequence ASTTTNYT-NH₂ can be of the D configuration. In some instances, the A in the sequence ASTTTNYT-NH₂ can be in the D configuration and all other amino acids in this sequence can be in the L configuration. In the sequence, the —NH₂ designations that the amino acid threonine in the C terminal end of the sequence has an amide as opposed to a carboxylic acid.

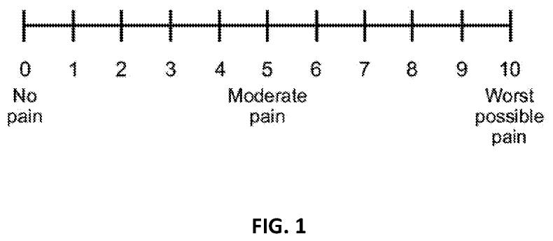

In some instances, the polypeptide sequence is a multi-chemokine receptor antagonist RAP-103 (R103) (All D-peptide-Thr-Thr-Asn-Tyr-Thr) as shown in FIGS. 1-4.

In some instances, the polypeptide described herein can be in the form of a pharmaceutically acceptable salt, such as acetate.

### Pharmaceutical Compositions

An active pharmaceutical ingredient may be any substance or mixture of substances intended to be used in the manufacture of a drug (medicinal) product and that, when used in the production of a drug, becomes an active ingredient of the drug product. Such substances may be intended to furnish pharmacological activity or other direct effect in the diagnosis, cure, mitigation, treatment, or prevention of disease or to affect the structure or function of the body.

### Representative Acids for Addition Salts

In some cases, the pharmaceutically acceptable salt of the polypeptide can be formed from the polypeptide and an acid. In some cases, the acid can be at least one of: 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p), undecylenic acid, or any combination thereof.

### Representative Salts

In some instances, the pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparaginate, glutamate and the like. In some instances, a salt of a polypeptide or derivative thereof or a compound can be a Zwitterionic salt.

In some aspects, the pharmaceutical composition comprising the salt of the pharmaceutically active ingredient, wherein the salt comprises an organic salt, an inorganic salt, or any combination thereof. In some cases, an organic salt may comprise a phosphinate (e.g., sodium hypophosphite), a hydrazinium salt, a urate, a diazonium salt, an oxalate salt, a tartrate, a choline chloride. An example of an inorganic salt may be sodium chloride, calcium chloride, magnesium chloride, sodium bicarbonate, potassium chloride, sodium sulfate, calcium carbonate, calcium phosphate, or any combination thereof. In some aspects, the pharmaceutical composition comprising the salt of the pharmaceutically active ingredient, wherein the salt comprises an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a borate salt, a bitartrate salt, a palmitic acid salt, a phosphoric acid salt, or any combination thereof.

### Representative Excipients

In some instances, a pharmaceutical composition can comprise a pharmaceutically acceptable: carrier, diluent, excipient or any combination thereof. In some embodiments, An excipient can refer to a substance formulated alongside the active ingredient of a medication, included for the purpose of long-term stabilization, bulking up solid formulations that contain potent active ingredients in small amounts, and/or to confer a therapeutic enhancement on the active ingredient(s) in the final dosage form. Excipients may facilitate drug absorption, reduce viscosity, or enhance solubility. Excipients may also facilitate the handling of the active ingredients, improve in vitro stability, and/or extend pharmaceutical product shelf life. Excipient selection may vary with the route of administration for drug delivery, the unit dose, as well as the active ingredients comprising the composition.

In some instances, a pharmaceutically acceptable excipient can comprise anhydrous calcium phosphate, dihydrate calcium phosphate, hydroxypropyl methylcellulose, croscarmellose sodium, GMO-free croscarmellose sodium, carbomers, magnesium aluminometasilicate, mannitol, povidone (PVP), crospovidone, sorbitol, dimethicone, sodium stearyl fumarate, sodium starch glycollate, hydroxypropylcellulose, native corn starch, modified corn starch, carrageenan, alginates, silicon dioxide, microcrystalline cellulose, carboxymethylcellulose sodium, alginates, carboxymethylcellulose (CMC), sodium carboxymethylcellulose (Na CMC), carbomers, natural gums, sorbitol, maltitol, glucose syrup, silicones, carbomers, fatty alcohols, alcohols, carbohydrates, petrolatum derivatives, butters, waxes, DMSO Procipient®, esters, fatty acids, oil-in-water (O/W) emulsifiers, water-in-oil (W/O) emulsifiers, silicas, fumed silicas, polysorbates, isopropyl myristate, cellulosic derivates, xanthan gum, propylenglycol, noveon AA-1 polycarbophyl, dimethyl isosorbate, polysilicone elastomer 1100, polysilicone elastomer 1148P, preservatives, flavors, colors, functional coatings, aesthetic coatings, a pharmaceutically acceptable salt of any of these, or any combination thereof.

In some cases, a pharmaceutically acceptable excipient can comprise acacia, acesulfame potassium, acetic acid, glacial, acetone, acetyl tributyl citrate, acetyl triethyl citrate, agar, albumin, alcohol, alginic acid, aliphatic polyesters, alitame, almond oil, alpha tocopherol, aluminum hydroxide adjuvant, aluminum oxide, aluminum phosphate adjuvant, aluminum stearate, ammonia solution, ammonium alginate, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylparaben, calcium alginate, calcium carbonate, calcium phosphate, dibasic anhydrous, calcium phosphate, dibasic dihydrate, calcium phosphate, tribasic, calcium stearate, calcium sulfate, canola oil, carbomer, carbon dioxide, carboxymethylcellulose calcium, carboxymethylcellulose sodium, carrageenan, castor oil, castor oil, hydrogenated, cellulose (e.g. microcrystalline, powdered, silicified microcrystalline, acetate, acetate phthalate) ceratonia, cetostearyl alcohol, cetrimide, cetyl alcohol, cetylpyridinium chloride, chitosan, chlorhexidine, chlorobutanol, chlorocresol, chlorodifluoroethane, chlorofluorocarbons, chloroxylenol, cholesterol, citric acid monohydrate, colloidal silicon dioxide, coloring agents, copovidone, corn oil, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane, dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol palmitostearate, ethylene vinyl acetate, ethylparaben, fructose, fumaric acid, gelatin, glucose, glycerin, glyceryl behenate, glyceryl monooleate, glyceryl monostearate, glyceryl palmitostearate, glycofurol, guar gum, hectorite, heptafluoropropane, hexetidine, hydrocarbons, hydrochloric acid, hydroxyethyl cellulose, hydroxyethylmethyl cellulose, hydroxypropyl cellulose, hydroxypropyl cellulose, low-substituted, hydroxypropyl starch, hypromellose, hypromellose acetate succinate, hypromellose phthalate, honey, imidurea, inulin, iron oxides, isomalt, isopropyl alcohol, isopropyl myristate, isopropyl palmitate, kaolin, lactic acid, lactitol, lactose, anhydrous, lactose, monohydrate, lactose, spray-dried, lanolin, lanolin alcohols, lanolin, hydrous, lauric acid, lecithin, leucine, linoleic acid, macrogol hydroxystearate, magnesium aluminum silicate, magnesium carbonate, magnesium oxide, magnesium silicate, magnesium stearate, magnesium trisilicate, malic acid, maltitol, maltitol solution, maltodextrin, maltol, maltose, mannitol, medium-chain triglycerides, meglumine, menthol, methylcellulose, methylparaben, mineral oil, mineral oil, light, mineral oil and lanolin alcohols, monoethanolamine, monosodium glutamate, monothioglycerol, myristic acid, neohesperidin dihydrochalcone, nitrogen, nitrous oxide, octyldodecanol, oleic acid, oleyl alcohol, olive oil, palmitic acid, paraffin, peanut oil, pectin, petrolatum, petrolatum and lanolin alcohols, phenol, phenoxyethanol, phenylethyl alcohol, phenylmercuric acetate, phenylmercuric borate, phenylmercuric nitrate, phosphoric acid, polacrilin potassium, poloxamer, polycarbophil, polydextrose, polyethylene glycol, polyethylene oxide, polymethacrylates, poly(methyl vinyl ether/maleic anhydride), polyoxyethylene alkyl ethers, polyoxyethylene castor oil derivatives, polyoxyethylene sorbitan fatty acid esters, polyoxyethylene stearates, polyvinyl acetate phthalate, polyvinyl alcohol, potassium alginate, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, 2-pyrrolidone, raffinose, saccharin, saccharin sodium, saponite, sesame oil, shellac, simethicone, sodium acetate, sodium alginate, sodium ascorbate, sodium benzoate, sodium bicarbonate, sodium borate, sodium chloride, sodium citrate dihydrate, sodium cyclamate, sodium hyaluronate, sodium hydroxide, sodium lactate, sodium lauryl sulfate, sodium metabisulfite, sodium phosphate, dibasic, sodium phosphate, monobasic, sodium propionate, sodium starch glycolate, sodium stearyl fumarate, sodium sulfite, sorbic acid, sorbitan esters (sorbitan fatty acid esters), sorbitol, soybean oil, starch, starch (e.g. pregelatinized, sterilizable maize), stearic acid, stearyl alcohol, sucralose, sucrose, sugar, compressible, sugar, confectioner's, sugar spheres, sulfobutylether b-cyclodextrin, sulfuric acid, sunflower oil, suppository bases, hard fat, talc, tartaric acid, tetrafluoroethane, thaumatin, thimerosal, thymol, titanium dioxide, tragacanth, trehalose, triacetin, tributyl citrate, triethanolamine, triethyl citrate, vanillin, vegetable oil, hydrogenated, water, wax, anionic emulsifying, wax (e.g. carnauba, cetyl esters, microcrystalline, nonionic emulsifying, white, yellow), xanthan gum, xylitol, zein, zinc acetate, zinc stearate, or any combination thereof.

In some cases, a pharmaceutically acceptable excipient can comprise a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof. In some cases, a pharmaceutically acceptable excipient can comprise a carbohydrate. In some cases, the carbohydrate can comprise lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof.

### Carriers and Diluents

In some instances, a pharmaceutically acceptable carrier or diluent can comprise water. In some cases, the water can be sterile. In some cases, the water can contain a buffer, a carbohydrate, a salt, a pH adjuster, or any combination of these. Simple sugars such as mannitol, sucrose, glucose, or trehalose may be added to inhibit peptide or polypeptide aggregation, in amounts from 1 to 50 mgs/ml. Citrate can be used as a buffer. In some instances, sodium chloride and phosphate salts may or may not be employed. Larger polysaccharides may also be used to enhance stability. In some cases, a diluent can comprise a saline solution.

In certain instances, a carrier may refer to reagents, cells, compounds, materials, compositions, dosage forms, or any combination thereof that can be compatible with agents that can be administered therapeutically. In some cases, a carrier can be suitable for use in contact with a tissue of a subject. In some cases, a carrier may not have a toxicity, an irritation, an allergic response, or any combination thereof. A carrier that may be suitable for use can include a liquid, a solid material (e.g., a pill, or a suppository) or any combination thereof. In some cases, a carrier can be designed to resist degradation within the body (non-biodegradable) or they may be designed to degrade within the body (biodegradable). A biodegradable material can further be bioresorbable or bioabsorbable. In some cases, a biodegradable material can be degraded and eliminated from the body by conversion into other materials or breakdown and elimination through natural pathways.

### Oral Bioavailability

In some instances, the polypeptides, derivatives thereof, or salts of any of these can be orally bioavailable. The percent oral bioavailability can be at least, at least about, or about: 5%, 10% 20%, 30%, 40%, 50%, 60% 70%, 80%, 90%, or 95%, or 100%.

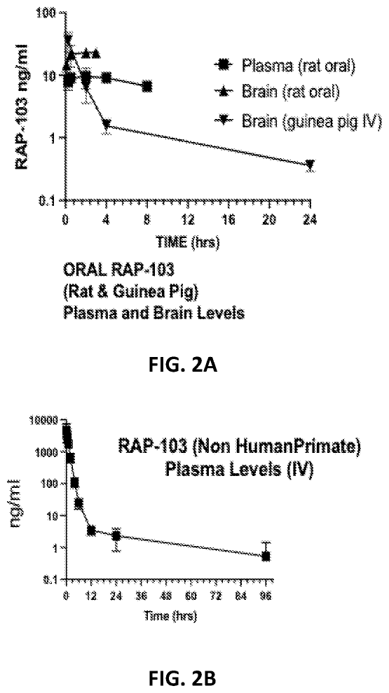

For example, in some studies, RAP-103 quickly entered the brain by oral, and IV dosing in rodents and non-human primates (Rhesus macaque). The non-human primates showed oral bioavailability of 88%. In some studies, RAP-103 preferentially entered the brain by oral compared to IV dosing, a feature that supports its use in the treatment of pain. (FIG. 2A).

In some instances, the polypeptides, derivatives, salts of any of these, can be administered to a subject, who can be a subject in need thereof. In some cases, the subject has PPSP. In some cases, the subject can be a human, can be a male, or can be a female. In some instances, the subject can be under 18 years of age. In some instances, the subject can be over 18 years of age. In some instances, the subject can range from about 6 months of age to about 120 years of age.

### Administration

In some aspects, the terms “administer,” “administering”, “administration,” and the like, as used herein, can refer to methods that can be used to enable delivery of compounds, polypeptides, derivatives thereof, or salts of any of these, or compositions described herein, to the desired site of biological action. In some cases, delivery can include injection, inhalation, catheterization, gastrostomy tube administration, intravenous administration, intraosseous administration, ocular administration, otic administration, topical administration, transdermal administration, local administration, oral administration, rectal administration, nasal administration, intravaginal administration, intracavernous administration, transurethral administration, buccal administration, sublingual administration, or a combination thereof. Delivery can include direct application to the affect tissue or region of the body. Delivery can include a parenchymal injection, an intra-thecal injection, an intra-ventricular injection, or an intra-cisternal injection. A composition provided herein can be administered by any method. A method of administration can be by intraarterial injection, intracerebroventricular injection, intracisternal injection, intramuscular injection, intraorbital injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some cases, delivery can comprise delivery of the peptides in the form of a pill, a capsule, or a liquid. In some instances, delivery can comprise a nanoparticle, a viral vector, a viral-like particle, a liposome, an exosome, an extracellular vesicle, a microrobot, a microneedle, an implant, or a combination thereof. In some cases, delivery can be from a device. In some instances, delivery can be administered by a pump, an infusion pump or a combination thereof. In some cases, delivery can be by an enema, an eye drop, a nasal spray, an ear drop, or any combination thereof. In some cases, delivery can comprise an inhaler, a diffuser, a nebulizer, or a combination thereof. Delivery can include topical administration (such as a lotion, a cream, a patch, a gel, a spray, a drip, a liquid formulation, an ointment) to an external surface of a surface, such as a skin. In some instances, a subject can administer the composition in the absence of supervision. In some instances, a subject can administer the composition under the supervision of a medical professional (e.g., a physician, nurse, physician's assistant, orderly, hospice worker, etc.). In some cases, a medical professional can administer the composition. In some cases, the subject can administer the composition.

In some embodiments, administering can be performed at least about: 1 time per day, 2 times per day, 3 times per day, 4 times per day, 5 times per day, 6 times per day or more than 6 times per day. In some cases, administering can be performed daily, weekly, monthly, or as needed. In some embodiments, administering can be conducted one, twice, three, or four times per day. In some cases, administration can be provided by a subject (e.g., the patient), a health care provider, or both.

Administration or application of a composition disclosed herein can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, or 1000 days consecutive or nonconsecutive days. In some cases, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, from about 29 to about 30 days, from about 1 to about 90 days, from about 30 day to about 90 days, from about 60 days to about 90 days, from about 30 days to about 180 days, or from about 90 days to about 190 days.

Administration or application of a composition disclosed herein can be performed for a treatment duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or for life. Administration can be performed repeatedly over a lifetime of a subject, such as once a month or once a year for the lifetime of a subject. Administration can be performed repeatedly over a substantial portion of a subject's life, such as once a month or once a year for at least about 1 year, 5 years, 10 years, 15 years, 20 years, 25 years, 30 years, or more.

Administration or application of a composition disclosed herein can be performed at least 1, 2, 3, 4, 5, 6, 7, 8, 9, 10, 11, 12, 13, 14, 15, 16, 17, 18, 19, 20, 21, 22, 23, or 24 times in a 24-hour period. In some cases, administration or application of a composition disclosed herein can be performed continuously throughout a 24-hour period, for example, when an implant can be used for administration. In some cases, administration or application of a composition disclosed herein can be performed at least 1, 2, 3, 4, 5, 6, 7, 8, 9, 10, 11, 12, 13, 14, 15, 16, 17, 18, 19, 20, or 21 times a week. In some cases, administration or application of a composition disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month. In some cases, a composition can be administered as a single dose or as divided doses. In some cases, the compositions described herein can be administered at a first time point and a second time point. In some cases, a composition can be administered such that a first administration can be administered before the other with a difference in administration time of 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

In some embodiments, administering can be performed for about: 1 day to about 8 days, 1 week to about 5 weeks, 1 month to about 12 months, 1 year to about 3 years, 3 years to about 10 years, 10 years to about 50 years, 25 years to about 100 years, or 50 years to about 130 years.

In some embodiments, a subject can be from about 1 day to about 10 months old, from about 9 months to about 24 months old, from about 1 year to about 8 years old, from about 5 years to about 25 years old, from about 20 years to about 50 years old, from about 40 years to about 80 years old, or from about 50 years to about 130 years old.

In some embodiments, the composition can be administered as needed, or for: one day, two days, three days, four days, five days, six days, a week, two weeks, three weeks, a month, two months, three months, four months, five months, six months, seven months, eight months, nine months, ten months, eleven months, a year, or chronically.

In some cases, the polypeptide or the derivative thereof or the salt of any of these can be administered in a pharmaceutical composition, which can be in unit dose form. In some instances, the amount of the polypeptide, or the derivative thereof, or the salt of any of these can be dosed in an amount ranging from about 0.0001 mg/kg of body weight of the subject to about 1000 g/kg of body weight of the subject; the dosage can be, for example, based on mg of polypeptide, derivative thereof, or salt thereof, per kg of subject body weight, can be about: 0.0001, 0.001, 0.01, 0.1, 0.2, 0.3, 0.4, 0.5, 0.6, 0.7, 0.8, 0.9 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, or 1000 g/kg of subject body weight.

In some instances, the amount of polypeptide, derivative thereof, or salt of any of these, which can be a pharmaceutically acceptable salt, that is dosed to the patient can range from 0.00001 mg to 1000 g; the dosage can be for example, about: 0.0001, 0.001, 0.01, 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, or 1000 g.

In some aspects, the composition may be administered so that the peptide, the derivative thereof or the pharmaceutically acceptable salt thereof is in an amount ranging from: 500 μg (micrograms) to about 1000 mg, 10 μg to about 50 μg, 40 μg to about 90 μg, 80 μg to about 120 μg, 100 μg to about 150 μg, 140 μg to about 190 μg, 150 μg to about 220 μg, 200 μg to about 250 μg, 240 μg to about 300 μg, 290 μg to about 350 μg, 340 μg to about 410 μg, 400 μg to about 450 μg, 440 μg to about 500 μg, 500 μg to about 700 μg, 600 μg to about 900 μg, 800 μg to about 1 mg, 1 mg to about 5 mg, 1 mg to about 10 mg, 5 mg to about 15 mg, 12 mg to about 25 mg, 20 mg to about 50 mg, 40 mg to about 80 mg, 70 mg to about 100 mg, 90 mg to about 150 mg, 125 mg to about 250 mg, 200 mg to about 500 mg, 400 mg to about 750 mg, 700 mg to about 900 mg, or from about 850 mg to about 1000 mg.

In some instances, a composition or pharmaceutical composition may be in the form of a capsule, a tablet, a gummy, an oil, a liquid, a tincture, a lotion, a cream, a balm, a candy, a chocolate, a food, a drink, an oil, a suppository, a liquid for injection, which can be, for example, an intra venous liquid, an intramuscular liquid, or subcutaneous liquid, a syrup or any combination thereof.

### Diagnoses

In some embodiments, a method can further comprise diagnosing a subject as having the disease. In some cases, the disease can be PPSP, a weight issue (such as obesity or underweight), or both. In some embodiments, a diagnosing can comprise employing an in vitro diagnostic. In some embodiments, the in vitro diagnostic can be a companion diagnostic.

In some embodiments, a diagnosis can comprise a physical examination, a radiological image, a blood, body fluid or tissue test, an antibody test, or any combination thereof. The diagnostic analyte can be a cytokine, such as a proinflammatory cytokine or a chemokine, or their receptors.

In some embodiments, a diagnosis can comprise a radiological image and the radiological image can comprise: a computed tomography (CT) image, an X-Ray image, a magnetic resonance image (MRI), an ultrasound image, or any combination thereof. Imaging markers of brain inflammation such as 18F-FEPPA, a TSPO ligand, may be used to support diagnoses or response to treatment. TSPO in some instances can mean translocator protein.

In some aspects, a method may further comprise diagnosing a subject as having the disease. In some aspects, a diagnosing may comprise employing an in vitro diagnostic. In some aspects, the in vitro diagnostic may be a companion diagnostic.

In some aspects, a diagnosis may comprise a physical examination, a radiological image, a blood test, an antibody test, or any combination thereof. In some aspects, a diagnosis may comprise a radiological image and the radiological image may comprise: a computed tomography (CT) image, an X-Ray image, a magnetic resonance image (MRI), an ultrasound image, or any combination thereof.

### Kits

Also disclosed herein are kits comprising the pharmaceutical composition contained at least in part in packaging. Also disclosed herein are methods of making kits comprising a pharmaceutical composition contained at least in part in packaging.

### Methods of Treatment

Also disclosed herein are methods of treating a disease comprising treating the disease or condition by administering a therapeutically effective amount of the pharmaceutical composition.

In certain aspects, the condition is a result of, associated with, or in anticipation of a medical procedure such as, but not limited to a thoracotomy, a cesarean section, a cardiac surgery, a breast surgery, an amputation, a total knee arthroplasty, a hip arthroplasty, a hernia repair, a cholecystectomy, a vasectomy, a cancer resection, a dental surgery or any combination thereof.

In some cases, the disease can be a weight associated disease such as obesity, overweight, or underweight. In some cases, a disease can be stress. In some cases, a disease can be depression. In some cases, a disease can be an ideation of suicide. In some cases, the method can be a method of treating pain catastrophizing with any one of the peptides disclosed herein.

In certain instances, the PPSP is due to an extracellular factor such as nerve growth factor (NGF) or brain-derived neurotrophic growth factor (BDNF).

### Co-Therapies

In some aspects, a method may further comprise administering a second therapy to the subject. In some aspects, a second therapy may comprise acetaminophen, an opioid, prednisone, cortisone, a gabapentinoid, a voltage gated sodium channel inhibitor, an anti-nerve growth factor, a salt of any of these, or any combination thereof. In some instances, the second therapy may comprise a nonsteroidal anti-inflammatory drug and the nonsteroidal anti-inflammatory drug may comprise naproxen, ibuprofen, acetaminophen, aspirin a salt of any of these, or any combination thereof. In some cases, a second therapy can be administered concurrently or consecutively with a peptide disclosed herein.

In some aspects, the composition may be administered as needed, or for: one day, two days, three days, four days, five days, six days, a week, two weeks, three weeks, a month, two months, three months, four months, five months, six months, seven months, eight months, nine months, ten months, eleven months, a year, or chronically.

In some aspects, the composition may be administered so that the amount of the active ingredient, (such as the active ingredient in the second therapy, or a peptide disclosed herein) or the pharmaceutically acceptable salt thereof in ranges from about: 500 μg (micrograms) to about 1000 mg, 10 μg to about 50 μg, 40 μg to about 90 μg, 80 μg to about 120 μg, 100 μg to about 150 μg, 140 μg to about 190 μg, 150 μg to about 220 μg, 200 μg to about 250 μg, 240 μg to about 300 μg, 290 μg to about 350 μg, 340 μg to about 410 μg, 400 μg to about 450 μg, 440 μg to about 500 μg, 500 μg to about 700 μg, 600 μg to about 900 μg, 800 μg to about 1 mg, 1 mg to about 5 mg, 1 mg to about 10 mg, 5 mg to about 15 mg, 12 mg to about 25 mg, 20 mg to about 50 mg, 40 mg to about 80 mg, 70 mg to about 100 mg, 90 mg to about 150 mg, 125 mg to about 250 mg, 200 mg to about 500 mg, 400 mg to about 750 mg, 700 mg to about 900 mg, or from about 850 mg to about 1000 mg. In some cases, the amount of the active ingredient or salt thereof may be more than about: 10 μg, 25 μg, 50 μg, 75 μg, 100 μg, 150 μg, 200 μg, 220 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 50 mg, 75 mg, 100 mg, 125 mg, 150 mg, 200 mg, 300 mg, 400 mg or 500 mg. In some cases, the amount of the active ingredient or salt thereof may be less than about: 10 μg, 25 μg, 50 μg, 75 μg, 100 μg, 150 μg, 200 μg, 220 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 50 mg, 75 mg, 100 mg, 125 mg, 150 mg, 200 mg, 300 mg, 400 mg or 500 mg.

### EXAMPLES

The following examples are included for illustrative purposes only and are not intended to limit the scope of the disclosure.

### Example 1

The synthetic process used for manufacture of RAP-103 Acetate Salt involves the following steps which follow the Merrifield FMOC synthesis method SPPS (Solid phase Peptide Synthesis), using the common commercially available coupling reagents, resins, and deblocking reagents:
Step 1: peptide synthesis: solid phase peptide synthesis (SPPS) of the protected peptide
Step 2: cleavage and deprotection: trifluoroacetic acid (TFA) cleavage of the protecting groups from the peptide and cleavage of the peptide from the resin
Step 3: purification and in-process lyophilization: peptide purification and in-process lyophilization of the peptide
Step 4: ion exchange (salt exchange) and final lyophilization: ion exchange (salt exchange) from TFA to acetate salt and Final lyophilization.

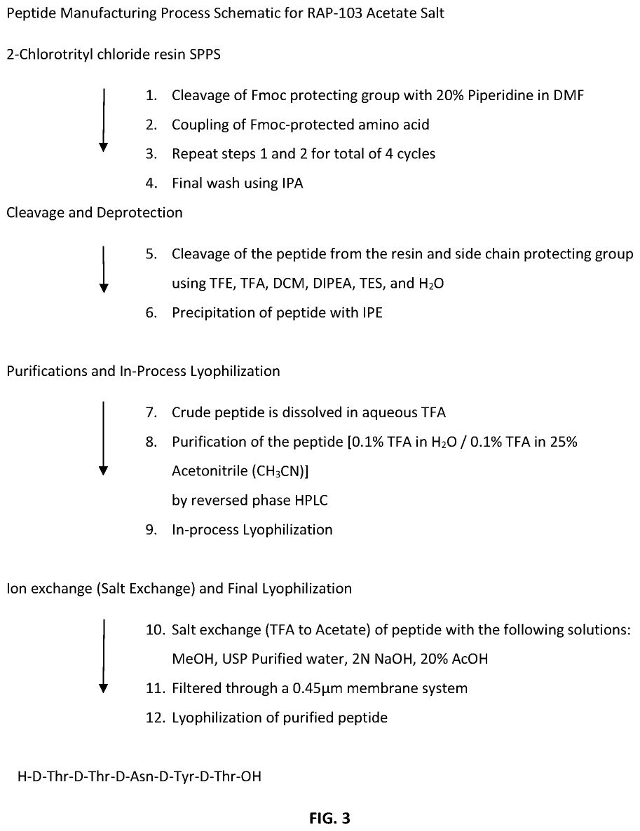

A schematic of a synthesis method for RAP-103 is shown in FIG. 3 and comprises the following steps: 2-chlorotrityl chloride resin SPPS, cleavage and deprotection, urifications and in-process lyophilization ion exchange (salt exchange) and final lyophilization. The following components are equipment and components were used for synthesis.

### Reaction Vessel

The synthesis was carried out at room temperature in a custom-designed glass vessel, with the bottom part comprising a fritted disk of coarse porosity. The size of the reactor is dependent on the amount of polymer to be used for the synthesis. The reactor was designed to assist in the addition of amino acid derivatives, solvents and reagents, as required. The reaction vessel was equipped with a mechanical stirrer to allow for efficient mixing of the peptide-resin. No components of the equipment or utensils utilized for the synthesis process were composed of materials that can cause adulteration of the product. Solid phase support: 2-Chlorotrityl chloride resin was used for the synthesis.

### Protected Amino Acids

In solid phase peptide synthesis, the reactive functional groups of the amino acids were protected to avoid undesirable side reactions. The protecting groups were of two natures: acid labile and base labile. The base labile protecting group was used to block the α-amino group during the coupling reaction and was removed in the deblocking step, to allow the introduction of the next amino acid in the sequence. Fmoc (9-Fluorenylmethyloxycarbonyl) was used as the base labile a-amino protecting group. The acid labile protecting group was used to protect the side-chain reactive functional groups of the amino acids during synthesis and must be resistant to the deblocking mixture (20% piperidine in DMF). Following the peptide synthesis, these protecting groups were removed by strong acid (aqueous trifluoroacetic acid with scavengers). The acid-labile protecting groups for this process are t-butyl (tBu), trityl (Trt).

The following amino acids were used in the synthesis of RAP-103 Acetate Salt: Fmoc-D-Tyr(tBu)-OH, Fmoc-D-Asn(Trt)-OH, and Fmoc-D-Thr(tBu)-OH.

### Step 1: Peptide Synthesis:

Resin Loading—2-Chlorotrityl chloride (CTC) resin was activated with Acetyl chloride (AcCl) and then treated with Fmoc-D-Thr(tBu)-OH and Diisopropylethylamine (DIPEA) followed by a solution mixture of Dichloromethane (DCM), Methanol (MeOH) and Diisopropylethylamine (DIPEA).

The solid phase peptide synthesis by Fmoc strategy can be divided into the following steps:

### Fmoc Deprotection:

During the deprotection step, the base-labile temporary protecting group (Fmoc) was cleaved from the α amino function of the N-terminal amino acid on the growing peptide chain by treating the resin twice with a solution of 20% piperidine in dimethylformamide (DMF). Two deprotection treatments were performed, the 1st deprotection stir time was approximately 10 minutes, and the 2nd deprotection stir time was approximately 30 minutes.

### Wash Cycle:

The wash steps were performed to eliminate excess reagents used in the preceding step. The solvents selected for each step were carefully chosen to ensure that there is no risk of introducing an undesirable side reaction while eliminating the excess of reagents as efficiently as possible. The duration of each wash step was timed to allow for thorough contact of the peptide-resin with the solvent and to provide ample time for extraction of the reagents. DMF was used after deblocking as well as after coupling because it has excellent solubilizing and swelling properties for all reagents used in the coupling step. Conversely, isopropanol (IPA) was utilized after coupling reaction because it shrinks the resin, which also aids in removal of excess solvents and reagents.

### Activation and Coupling:

During the activation and coupling steps, the deprotected a-amino group is acylated by the next activated amino acid in the sequence. The reagents used to accomplish acylation were carefully selected to create optimal reaction conditions and easy elimination of the excess reagents at the end of the coupling reaction.

Activation of Fmoc-Tyr(tBu)-OH was performed by dissolving the protected amino acid with coupling s 1-H-Benzotriazolium, 1-[bis(dimethylamino)methylene]-5-chloro-tetrafluoroborate(1-),3-oxide (TCTU) and diisopropylethylamine (DIPEA) in DMF. The solution of activated amino acid was then added to the peptide-resin. The mixture was stirred at room temperature for 20 minutes and then DIPEA in DMF was added in it. The mixture was allowed to react for approximately 160 minutes.

Activation of the remaining amino acid derivatives was performed by dissolving the protected amino acid with coupling reagents Oxima (Oxymapure) in DMF and 1,3-Diisopropylcarbodiimide (DIC). The solution of activated amino acid was then added to the peptide-resin. The suspension was stirred at room temperature for 20 minutes, after which a second aliquot of DIC was added to the reaction mixture. The mixture was stirred and allowed to react for approximately 160 minutes.

### Recoupling and Acetylation:

After a minimal reaction time of one hour, the presence of remaining unreacted amino groups was monitored using the qualitative TNBS (trinitrobenzenesulfonic acid) test or the Ninhydrin test. The TNBS test is performed adding a few drops of trinitrobenzenesulfonic acid to the peptide-resin in a test tube sample and allowing the two to react for three minutes. The presence of free amino groups causes a colored reaction; orange-colored beads indicate incomplete coupling and the presence of unreacted amine. Similarly, in the Ninhydrin test, a few drops of the Ninhydrin reagents were added to a sample of the peptide-resin in a small test tube. Blue-stained resin beads indicate the presence of unreacted amine.

If some residual amino groups were detected by either of the above-mentioned tests, the coupling reaction was repeated using half the amount of amino acid derivative required for the first coupling reaction. The Ninhydrin test was performed each time coupling takes place to visualize the presence of unreacted a-amino functions. No recoupling reactions were required during the manufacture of RAP-103 Acetate Salt lot 1000008388.

If unreacted a-amino functions are still present after recoupling, they are acetylated using acetic anhydride to avoid undesirable deletion sequences in the next cycle. No acetylation reactions were required during the manufacture of RAP-103 Acetate Salt lot 1000008388.

After coupling of the last amino acid in the sequence was completed, the peptide-resin was thoroughly washed using Isopropyl Alcohol (IPA) and weighed.

### Step 2: Cleavage and Deprotection:

During the cleavage operation, the peptide was detached from the resin with concomitant cleavage of the side chain protecting groups. This was accomplished by the treatment of the peptide-resin with trifluoroacetic acid (TFA) in the presence of scavengers and Trifluoroethanol (TFE) in TFA and Dichloromethane (DCM). Triethylsilane (TES) and water acted as scavengers and were used to provide a protonated cleavage environment which in turn gives higher quality crude. Following the cleavage operation, the peptide was precipitated using cooled isopropyl ether (IPE), and filtered using a Buchner funnel with filter paper. The precipitated peptide was washed with IPE and dried in a vacuum oven at room temperature. After drying was completed, the crude peptide was weighed and recorded.

### Step 3: Purification and In-Process Lyophilization:

The purification is performed by preparative Reversed Phase High Performance Chromatography (RP-HPLC).

### Equipment:

The purification equipment was based on the principle of compression in which the chromatographic support is packed in a compression module. A constant pressure was applied to the column. Different column sizes are available and the choice of which to use is based on the amount of material to be processed. The solvents were delivered through pumps and the necessary gradients are created manually or with an automatic gradient maker.

### Nature of Support:

The purification of crude peptide was accomplished by preparative HPLC using reversed phase material as the support. The reversed phase material comprises a silica gel coated with aliphatic chains; the free remaining silanol groups have been end-capped to avoid undesirable ionic interaction/binding between the mixture to be purified and the support. The separation was based on the hydrophobic interaction between the peptide and the resin support. The use of different buffer systems in subsequent purification steps also improves the separation efficiency.

### Purification Using an Aqueous TFA/Acetonitrile Buffer Gradient:

A typical purification run comprises three steps: equilibration Luna C18 column, loading and elution of the product, and washing of the column to prepare it for the next run. Equilibration of the column was accomplished by washing it with aqueous TFA solution. The crude peptide was dissolved in an aqueous TFA solution, filtered and then was loaded onto the column. Product elution was achieved using a gradient of aqueous TFA and acetonitrile (CH3CN) buffer solutions. After product elution, the column was washed with aqueous acetonitrile to check the absence of product. The quality of each different fraction that were collected as the peptide elutes from the column was monitored by analytical HPLC. The fractions, which met the acceptance criteria for purity, were pooled as the main pool and proceed to the next step.

### In-Process Lyophilization:

The main pool of the product from the TFA purification step was filtered through a 0.45 μm membrane filter and lyophilized.

### Step 4: Ion-Exchange (Salt Exchange) and Final Lyophilization:

ion-exchange (salt exchange):

The Ion-exchange, also referred to as the salt exchange stage, converts the peptide into the required salt form (acetate salt). The preparation of ion-exchange resin (AMBERLITE IRN 78) was accomplished by washing the resin sequentially with methanol (MeOH), USP Water, 2N sodium hydroxide (NaOH), USP water, acetic acid (AcOH—20% in USP water) and USP water until neutrality. The peptide from in-process lyophilization step was dissolved in USP water and loaded onto already prepared Ion-exchange resin. After circulating for two hours, the peptide solution was eluted and collected in fraction collecting bottles. All fractions that met the establish criteria after analyzing with HPLC were collected and pooled together.

### Final Lyophilization

The main pool of the peptide solution from ion-exchange (salt exchange) step was filtered through 0.45 μm membrane filtration cap. The resulting filtrate (peptide solution) was lyophilized to obtain a bulk RAP-103 acetate salt peptide.

### Example 2

A peptide described herein can be administered by a pill or a capsule to a subject in need thereof. The pills or capsules contain excipients to enhance stability, dissolution, and absorption. Enteric coatings are applied to control delivery and maintain therapeutic levels. In another other example, liquid solutions in water or saline are prepared for IV, sub-cutaneous, or intra-muscular delivery. Reconstitution at the time of use extends the shelf-life. The weight/weight ratio of drug (active pharmaceutical peptide) to excipient can be 0.01 to 0.25. In some cases, the weight/weight ratio of drug (active pharmaceutical peptide) to excipient can be 0.005 to 0.5.

### Example 3

Animals (rats, guinea pigs, and non-human primates (Rhesus macaques; Macaca mulatta) were administered RAP-103 by oral gavage and IV routes. Blood samples were collected into K2EDTA MAP tubes, placed on wet ice, and processed to plasma (in a centrifuge set to maintain 2000 g, at 4ºC for 15 minutes) within 60 minutes of collection and were stored in a freezer set to maintain −80° C. until analysis. Samples were analyzed by using a qualified high performance liquid chromatography (HPLC) with mass spectrometric (MS/MS) detection to determine the concentrations of RAP-103. Giving dosage forms to animals shows that RAP-103 quickly entered the brain (rats and guinea pigs) by IV or oral gavage dosing and persisted at therapeutic levels for at least 24 hrs as shown in FIG. 2A. RAP-103 preferentially enters the brain compared to plasma levels. In non-human primates (rhesus monkeys) RAP-103 was dosed once on Day 1 by intravenous bolus injection at 1 mg/kg and plasma levels determined. The Cmax and AUC 0-T were comparable in male and female monkeys. RAP-103 was highly bioavailable at the 1 mg/kg dose level with absolute bioavailability value of 88% and 89% in females and males. Drug in plasma after a single IV dose (1 mg/Kg) was still measurable at anticipated therapeutic levels 96 hrs. post-dose in non-human primates as shown in FIG. 2B. There were no RAP-103-related changes noted in clinical observations or body weights over seven days indicating no acute toxicity.

### Example 4

Chronic pain development was determined with behavior and molecular/cellular testing and inflammatory reactions in Streptozotocin (STZ)-induced diabetic rats treated with RAP-103.

Induction and assessment of diabetes in rats: Rats were fasted overnight and given a single intraperitoneal injection of STZ (60 mg/kg, Sigma-Aldrich) in citrate buffer (pH 4.5) to induce type I diabetes. The induction of diabetes was confirmed 72 h post-injection by blood glucose levels (Contour® blood glucose diagnostic kit, Bayer HealthCare, Canada). Body weight and blood glucose levels were measured before and 3 days post-STZ injection and once a week for three weeks post-diabetes induction. Only rats with blood glucose concentration consistently higher than 300 mg/dl were used for the study. Rats used for the study were monitored for persistent mechanical and cold allodynia (up to five months post-induction) and only animals with persistent pain were used.

Drug preparations: RAP-103 (98.8% purity, Bachem) was prepared in autoclaved H2O at room temperature, resulting in a 0.1 mg/ml solution (5 mg in 50 ml). This solution was prepared freshly for each experiment and kept for 8 days (duration of one experiment) at room temperature. Appropriate concentration for each dose was adjusted accordingly.

Treatment paradigms: Autoclaved water (vehicle) or RAP-103 (0.004, 0.02, 0.1 or 0.5 mg/kg b.w.) was administered daily for 8 days by oral gavage to rats with stable mechanical and cold allodynia (n=5 H2O; n=7 RAP-103).

Assessment of neuropathic pain: Tactile allodynia, mechanical sensitivity was assessed using calibrated. Animals were placed in plexiglass boxes on an elevated metal mesh floor and allowed 60 min for habituation before testing. A series of von Frey filaments with logarithmically incrementing stiffness (Stoelting) was applied perpendicular to the midplantar region of the hind paw. The 50% paw withdrawal threshold was determined using Dixon's up-down method. Withdrawal thresholds of both paws were averaged as one single value per animal.

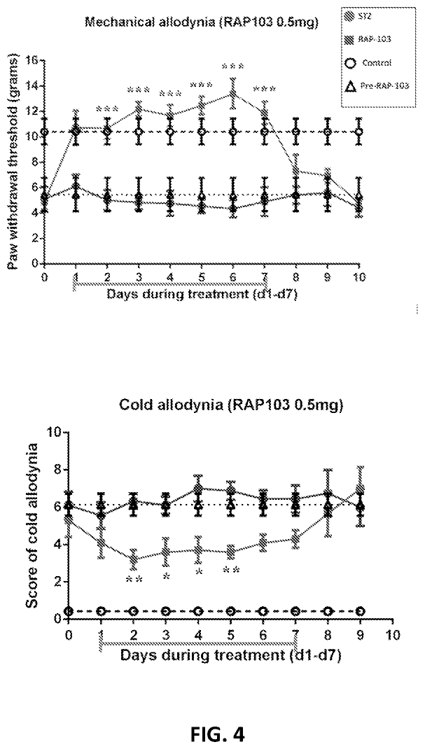

Cold Allodynia: was assessed using the same apparatus as the von Frey test. Rats were allowed to adapt to the testing environment for at least 10 min. Then, a drop (50 μl) of acetone was applied with a glass syringe fitted with a blunted needle at the centre of the plantar face of a hind paw. Responses were monitored during 1 min after acetone application and were graded according to a 4-point scale: 0, no response; 1, quick withdrawal, flick or stamp of the paw; 2, prolonged withdrawal or repeated flicking of the paw; 3, repeated flicking of the paw with persistent licking directed at the ventral side of the paw. Cumulative scores were then obtained by summing the four scores for each rat, the minimum score being 0 (no response to any of the four trials) and the maximum possible score being 12 (repeated flicking and licking of paws on each of the four trials). Rats were habituated to the testing environment. All animals were assessed for mechanical allodynia and cold allodynia of both hind paws before (behavioral baseline values before STZ injection) and once a week after diabetes induction until they exhibited stable hypersensitive states (before RAP-103 treatment), where the treatment with RAP-103 started. Assessment of RAP103 on mechanical and cold allodynia was performed between 2-4 hours following the drug administration and is shown in FIG. 4. All data is presented as means±SEM. Statistical analysis was performed by two-way ANOVA followed by Bonferroni post-tests. The criterion for statistical significance was p<0.05.

RAP-103 treatment reversed mechanical allodynia in STZ-induced diabetic rats. Following the induction of diabetes, rats exhibited persistent mechanical hyperalgesia up to five months post-induction. Daily oral gavage administration of RAP-103 resulted in a complete reversal of established hypersensitivity in STZ rats, which became significant within 24-48 hrs. RAP-103 doses of 0.5, 0.1, 0.02 mg/kg were effective, but not 0.004 mg/kg. FIG. 4 reports the result for the 0.5 mg/Kg exposure and may be representative of the other effective doses.

### Example 5

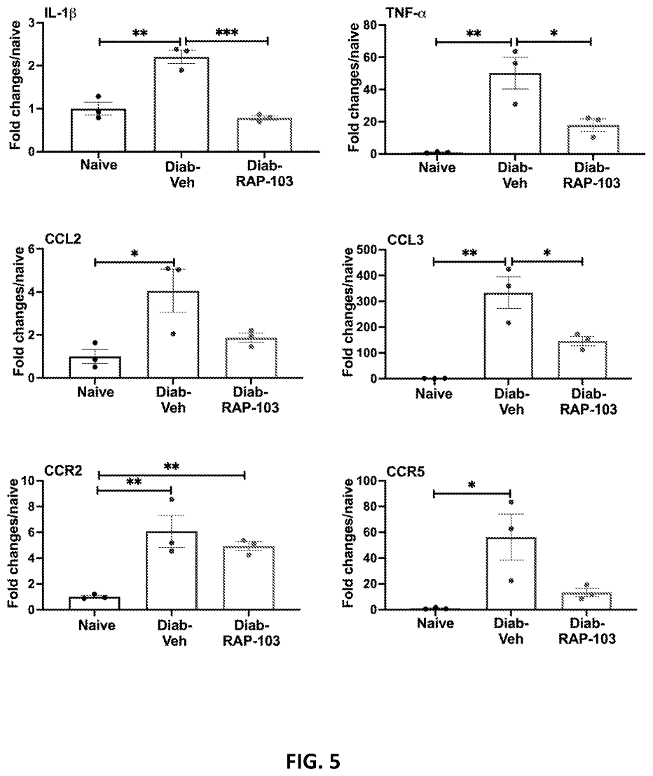

RAP-103 treatment suppressed cytokines and chemokines in sciatic nerves and/or spinal cord associated with persistent pain. Both IL-1β and TNFα were reduced after partial sciatic nerve ligation and biomarkers which contribute to or are associated with PPSP are reduced by RAP-103. IL-1β, TNFα and CCL3 were significantly reduced in nerve damage by partial nerve ligation and diabetic pain. Chemokines receptors CCR2 and CCR5 were lowered in neuropathic pain as shown in FIG. 5.

Pain after surgery can be a very specific entity as it may not be neither the result of an inflammatory process alone, nor it may not be only the result of isolated injury to nerves. The peptides and salts thereof can normalize dysregulated cytokines and chemokines as well as repair nerve injury, such as by promoting growth cone formation required for neurite extension that can reverse Wallerian degeneration or repair severed nerve connections that that occur in PPSP and incite pain. Blocking CCR5 and CXCR4 promotes nerve repair and the peptides and salts thereof can share this activity to deliver pain benefits in PPSP. Total RNA was extracted from sciatic nerves with RNeasy lipid tissue mini kit reported. (Qiagen, Toronto, ON, Canada or Valencia, CA). Synthesis of cDNA from total RNA was performed with SuperScript VILO cDNA synthesis kit (Invitrogen). Primers were produced by Qiagen QuantiTect. Sciatic nerves collected from the following groups were analyzed: 3 naive animals; 3 nerve injured H2O-treated animals, and 4 nerve injured RAP-103-treated animals. Experiments were performed in triplicates. Levels of target mRNAs were normalized to the housekeeping gene GAPDH. Fold changes versus naive animals were analyzed by using the comparative Ct (dCT) method. Detection of mRNAs encoding CCL2, CCL3, CCR2, CCR5, IL1β, and TNFα was performed as shown in FIG. 5.

### Example 6

The specific cytokines IL-1β, IL-10 and IL-12 have been identified as imbalanced following total knee replacement surgeries which increases risk for persistent post-operative pain. Measurement of these cytokines may be used to predict response to therapy, such as treatment by any of the peptides, salts thereof, or compositions of these, including those herein. Since the administration of the peptide DAPTA normalizes multiple plasma cytokines associated with PPSP then DAPTA and other analogs as we have described may be used to treat chronic pain conditions, specifically PPSP. Five HIV patients received DAPTA, 3 mgs/day, by metered nasal spray for 6 to 8 weeks (mean 7+0.6 wks). Patients were not taking other concurrent anti-pain or anti-retroviral therapies. At the beginning and end of the study period heparinized blood was collected and immediately clarified by centrifugation. Plasma aliquots were made and stored frozen at −70 C° for further testing. All sample testing was done on blinded samples at independent testing centers.

Cytokine measurements in patients treated with DAPTA were made after capillary electrophoresis isolation of individual cytokine peaks, followed by enzyme-linked immunoassay using specific antibodies. In the capillary electrophoresis method the analytes separate due to their electrophoretic mobility, and are detected at the outlet end of the capillary by the detector which then displays an electropherogram, a report of detector response as a function of time. Separated chemical compounds appear as peaks with different migration times in an electropherogram. The purified sample peaks were individually collected and concentration determined by a quantitative enzyme-linked immunosorbent assay (ELISA) using specific antibodies to determine the analyte concentration. The data are presented in Table 3, which shows that the close analog of RAP-103, the peptide DAPTA, causes changes in plasma cytokines in humans in the treatment of persistent post-surgical pain and other chronic pain conditions.

TABLE 3
Cytokine measurements in patients treated with DAPTA

| | CYTOKINE MEAN | | % CHANGE |
|---|---|---|---|
| | pgs/ml ± s.e.m. | | BASELINE to POST- |
| CYTOKINE | Baseline | Post-Treatment | DRUG (**p < .005) |
| TNFα | 339 ± 86 | 260 ± 67 | −23%** |
| IL-1β | 320 ± 85 | 174 ± 26 | −46%** |
| IL-6 | 179 ± 15 | 130 ± 21 | −28%** |
| IL-8 | 207 ± 68 | 144 ± 76 | −30%** |
| IL-2 | 14 ± 6 | 57 ± 9 | +395** |
| IL-4 | 6 ± 2 | 28 ± 8 | +448** |
| IL-10 | 13 ± 2 | 45 ± 5 | +350** |
| IL-12 | 9 ± 3 | 33 ± 7 | +363** |
| IL-13 | 7 ± 3 | 30 ± 9 | +416** |

Following intranasal DAPTA dosing significant reductions in the plasma levels of IL-1β, IL-6, IL-8, and TNFα were observed (P<0.005). The cytokines IL-4, IL-10, IL-12 and IL-13 were increased significantly (P<0.005). Where significant changes occurred (increase or decrease), all five members of the cohort responded in the same direction. Lowering the plasma levels of IL-1β, IL-6, IL-8, and TNFα and/or elevating the plasma levels of the cytokines IL-2, IL-4, IL-10, IL-12 and IL-13, individually or collectively or in any combination will reduce or prevent PPSP from occurring and potentially provide a treatment for persistent pain after surgery.

### Example 7

### RAP-103 Enhances Morphine Analgesia in Post-Surgical Pain.

Opioid receptor desensitization can contribute to PPSP and enhancement of opioid receptor pain responses would contribute to a PPSP pain benefit. The effect of RAP-103 on morphine analgesia after a surgical injury to the paw was determined.

Rats were acclimated for 30 min on the day of surgery and then their individual baseline values for paw withdrawal threshold were measured using a series of von Frey filaments (North Coast Medical, Inc., Gilroy, CA). We determined the force (gradually increasing equal logarithmic bending forces between 2-60 g), starting with the lowest, that causes paw withdrawal by applying force perpendicularly to the paw of the rat. Each filament was tested five consecutive times 10 seconds apart. A positive response was defined as quick withdrawal or paw flinching after the application of a filament.

Surgery was performed under isoflurane anesthesia (4% isoflurane for induction and 2.5% isoflurane for maintenance of anesthesia) in aseptic conditions. A 1-cm longitudinal incision (starting 0.5 cm from the proximal edge of the heel and extending toward the toes) was made with a scalpel through skin and the fascia of the plantar side of the hind paw. The plantaris muscle was exposed, elevated and incised longitudinally. Following bleeding control with gentle pressure, the skin was closed with two single interrupted sutures using 5-0 nylon. Animals were brought back to the individual chambers for mechanical allodynia testing. The time when surgeries were completed was designated as time 0. The animals awoke on the average approximately 5-8 min after the end of the surgery, and fully regained consciousness by the first testing time, which was 15 min post-surgery. Paw withdrawal thresholds were recorded at time points post-surgery (15, 30, 45, 60, 120, 240, and 360 min; 24, 48, and 72 hr) by a person blinded to the treatments. Morphine or the saline vehicle were injected subcutaneously (s.c.), and RAP-103 or the vehicle (water) for were given i.p., at t=25 min post-surgery.

For the surgical pain studies, data for morphine, RAP-103 time-course and dose-response curves, as well as data on the combination of morphine with RAP-103 were analyzed using two-way analysis of variance (ANOVA) followed by Sidak's multiple comparison tests for comparison of both RAP-103 and saline (control) time and treatment effects. To establish ED50 values and 95% confidence limits for morphine, for the combination of morphine with RAP-103 data from dose-response curves measuring percent reversal of mechanical allodynia were used and analyzed by nonlinear regression analysis. To compare dose-response curves for morphine alone and in combination with RAP-103, the paired Student's t-test was used. Data are expressed as mean±standard error of the mean (S.E.M.), and p<0.05 was accepted as statistically significant.

Co-Administration of RAP-103 with Morphine Enhances Analgesia.

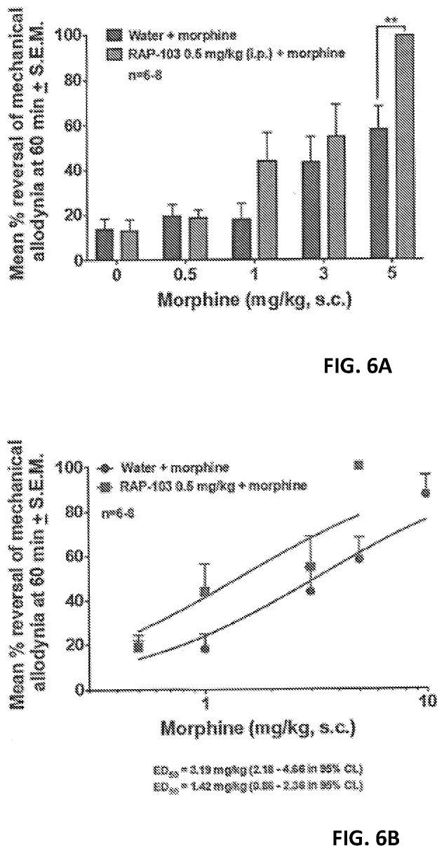

RAP-103 at a single dose, 0.5 mg/kg, i.p. was combined with sub-optimal doses of morphine (0.5 to 5.0 mg/kg) and the percent reversal of mechanical allodynia was compared to that of morphine alone. Since the maximal effect with morphine was established at 60 min post-surgery, percent reversal of mechanical allodynia was calculated for all groups at this time point. RAP-103 exhibits significant plasma bioavailability by oral, i.p., ss, and IV dosing with half-life (HL) elimination values of approximately 2.7 hours and thus would be at near maximal levels during the period of peak morphine effect. As shown in FIG. 6A, co-administration of RAP-103 at 0.5 mg/kg with morphine at 5 mg/kg significantly increased the analgesic effect compared to morphine alone (5 mg/kg). The effect of RAP-103 was to increase the potency of morphine by over 2-fold, from EC50 3.19 mg/Kg to 1.42 mg/Kg (FIG. 6B). RAP-103 is therefore demonstrated to enhance μ-opioid receptor analgesic activity. The effect would promote anti-nociceptive responses via endogenous μ and κ opioid receptor peptides after surgeries and contribute to the alleviation of PPSP.

### Example 8

### RAP-103 Restores Synapses Caused by Nerve Damage

In order to determine the ability of RAP-103 to reverse nerve damage and promote re-innervation, we studied the effect of RAP-103 to restore synapses damaged by treatment with aggregated amyloid beta peptide, which has been studied for its damaging effects on neuronal spines and synapses. Nerve damage by aggregated amyloid beta peptide is mediated via a common stress response involving mitochondrial damage and reactive oxygen species which also exist for nerves that are physically damaged, as occurs after a surgery.

AB dimer/trimer (d/t) was isolated by gel filtration of concentrated 7PA2 cell conditioned medium. Embryonic E16.5 mouse hippocampal neurons were plated on poly-D-lysine coated glass coverslips in 24 well plates and grown for 6 days in complete neurobasal medium. Cultured hippocampal neurons were maintained at 37° C. in a 5% CO2-humidified incubator. Treatments were started at 11 DIV and fixation and immunolabeling was performed on DIV 14, resulting in 3 days of exposure to Aβd/t, RAP-103 or both.

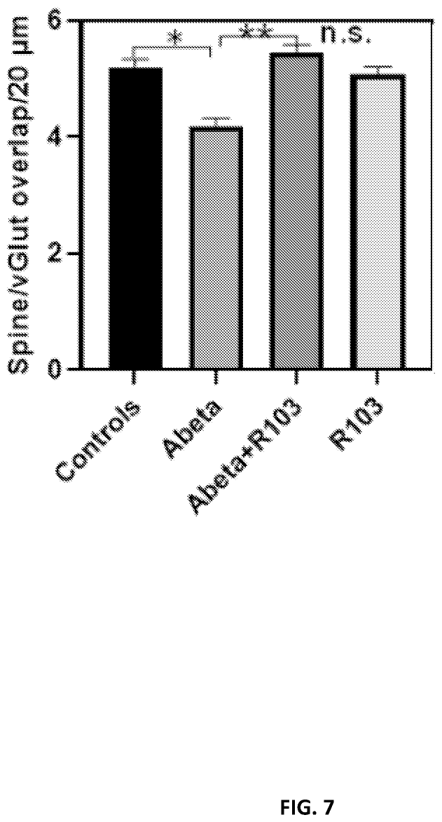

To determine synapse numbers, confocal images of mouse hippocampal neurons treated with vehicle or RAP-103 (0.1 nM) were fixed and stained for PSD95 (postsynaptic marker, green) and vGlut (presynaptic marker of excitatory glutamatergic synapses, red). Phase-contrast and fluorescence images were obtained on an inverted Nikon (Tokyo, Japan) TE 2000-S microscope using a 40× objective. Images were captured with a Hamamatsu (Hamamatsu City, Japan) ORCA 100-cooled CCD camera driven by Simple PCI software (Hamamatsu). Confocal images were collected on an Olympus FV1000 confocal microscope. Fluorescent images were processed with Adobe Photoshop software. For a synapse to be counted, the vGlut and PSD95 immunolabels needed to overlap or be directly abutted to each other. Results of the synapse and spine quantification are in FIG. 7 and indicate that RAP-103 has a synapse regenerating effect which we propose as a mechanism for a pain benefit in PPSP.

### Example 9

A subject is diagnosed with stress from PPSP. A subject is prescribed a dosing regimen of a pharmaceutical composition. The pharmaceutical composition comprises a therapeutically effective amount of RAP-103. RAP-103 is administered orally as a pill to treat the stress from PPSP. After daily administration for one week, stress and pain is reduced in the patent suffering from PPSP.

### Example 10

A subject is diagnosed with obesity. The subject is prescribed a dosing regimen of a pharmaceutical composition. The pharmaceutical composition comprises a therapeutically effective amount of RAP-103. RAP-103 is administered orally to treat obesity. After daily administration for 3 weeks the subject's weight decreases.

### Example 11

RAP-103 and related D-peptides disclosed herein are measured in a binding affinity assay to test their binding affinity to CX3CR1 receptor. Surface plasmon resonance (SPR) is used to test the binding affinity of RAP-103 and related peptides to the CX3CR1 receptor. Briefly, the CX3CR1 receptor is immobilized on a sensor chip and RAP-103 and related peptides are tested for their ability to bind to the receptor by a flow channel running the peptides over the receptor. SPR is detected in real time to determine the binding affinity. Additionally, Binding is assessed on intact cells using labeled CX3CL1 in a typical competition binding assay, or by functional inhibition of a CX3CL1 response, such as chemotaxis.

### Example 12

### Peptides and Chemotaxis Assay

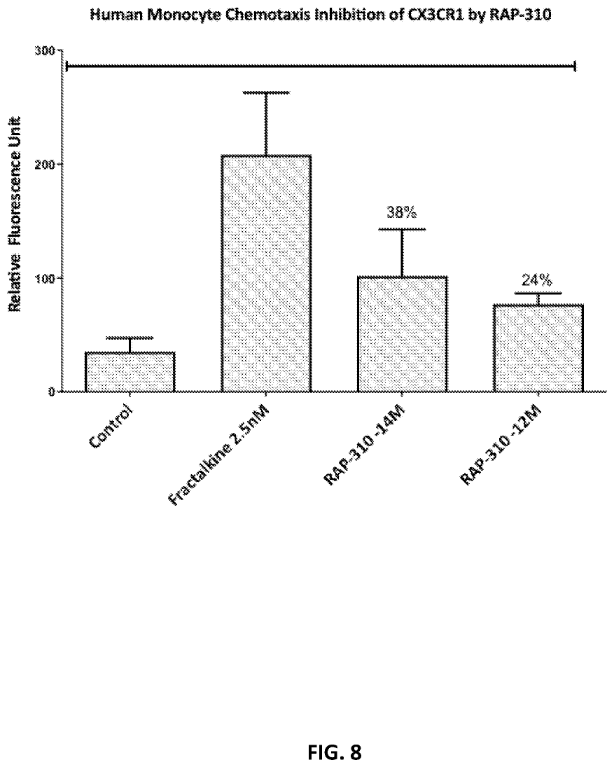

RAP-310 (all-D-ASTTTNYT) was synthesized by American Peptide Company, Inc., Sunnyvale, CA. All peptides were purified to >95% homogeneity and verified by HPLC isolation, amino acid analysis, and mass spectroscopy. Peptides were dissolved in sterile water and stored as frozen (−20° C.) aliquots at 0.1 mM until use. Chemotaxis was assayed in 96-well plates (NeuroProbe, Cabin John, MD) with 5 μM pore size, PVP-free membranes. Purified human monocytes (>95%) prepared from healthy adult human donors by centrifugal elutriation (>95% pure) were resuspended in chemotaxis assay buffer (DMEM supplemented with 0.1% BSA) at a density of 2×10⁶ cells/ml. Cells were labelled with 1.0 μM Calcein AM (Invitrogen) for 30 minutes at 37° C., 5% CO2. Following incubation, cells were washed once and resuspended in chemotaxis assay buffer (DMEM, 1 mg/ml BSA, 25 mM Hepes) at a density of 2×10⁶ cells/ml. Lower wells of the chemotaxis plate were filled with either chemotaxis assay buffer (control) or CX3CL1 (2.5 nM) as test chemoattractant with and without the indicated RAP-310 concentrations. The filter plate (5 μM pore size, PVP-free membrane) was snapped on over the filled wells and the monocytes were loaded onto the upper filter surface (75,000 cells in 25 μl). Chambers were then incubated at 37° C. for 90 minutes in humidified conditions. At the conclusion of the test period, non-migrating cells were wiped off the upper filter surface and relative fluorescence units (RFUs) of the migrating cells from the lower surface determined by bottom reading in a spectrometer (M5 SpectraMax) at 485/530 nm (Ex/Em). Triplicate determinations were made and results are expressed as the mean Relative Fluorescence Units as depicted in FIG. 8. Similar results were observed with RAP-103.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the methods presented in the disclosure. It is intended that the following claims define the scope of the disclosure and that methods and structures within the scope of these claims and their equivalents be covered thereby.

What is claimed is:

1. A method of inducing weight gain in a subject in need thereof, inducing weight loss in a subject in need thereof, or inducing a healthy weight in a subject in need thereof, the method comprising: administering to the subject a therapeutically effective amount of a pharmaceutical composition to induce weight gain in the subject in need thereof, induce weight loss in the subject in need thereof, or to induce healthy weight in the subject in need thereof, wherein the pharmaceutical composition comprises:
a polypeptide, a derivative thereof, or a salt thereof, wherein the polypeptide comprises at least five contiguous amino acids or derivatives thereof comprising the general formula: E-F-G-H-I, wherein:
E is D-Ser, D-Thr, D-Asn, D-Glu, D-Arg, D-Ile, or D-Leu, or a derivative of any of these;
F is D-Ser, D-Thr, D-Asp, or D-Asn, or a derivative of any of these;
G is D-Thr, D-Ser, D-Asn, D-Arg, D-Gln, D-Lys, or D-Trp, or a derivative of any of these;
H is D-Tyr, or a derivative thereof; and
I is D-Thr, D-Ser, D-Arg, or Gly, or a derivative of any of these.

2. The method of claim 1, wherein the polypeptide, the derivative thereof, or the salt thereof comprises at least eight contiguous amino acids or derivatives thereof, comprising the general formula A-B-C-E-F-G-H-I, and wherein:
A is D-Ala, or a derivative thereof;
B is D-Ser, or D-Thr, or a derivative of any of these;
C is D-Ser, or D-Thr, or a derivative of any of these;
E is D-Ser, D-Thr, D-Asn, D-Glu, D-Arg, D-Ile, or D-Leu, or a derivative of any of these;
F is D-Ser, D-Thr, D-Asp, or D-Asn, or a derivative of any of these;
G is D-Thr, D-Ser, D-Asn, D-Arg, D-Gln, D-Lys, or D-Trp, or a derivative of any of these;
H is D-Tyr, or a derivative thereof; and
I is D-Thr, D-Ser, D-Arg, or Gly or a derivative of any of these.

3. The method of claim 1 or claim 2, wherein the polypeptide or the salt thereof is D-Thr, D-Thr, D-Asn, D-Tyr, and D-Thr or a salt thereof.

4. The method of any one of claims 1-3, comprising the derivative of I, wherein I is esterified, glycosylated, or amidated at the C terminus.

5. The method of any one of claims 1-4, wherein the pharmaceutical composition is in unit dose form.

6. The method of any one of claims 1-5, wherein the pharmaceutical composition further comprises a pharmaceutically acceptable: excipient, diluent, carrier, or a combination thereof.

7. The method of any one of claims 1-6, wherein the method further comprises blocking neuropathic pain, enhancing opioid receptor analgesia, and promoting repair of neurons in the subject in need thereof.

8. The method of any one of claims 1-7, wherein the method further comprises blocking ligand binding to a CX3CR1 receptor, reducing stress, or both in the subject in need thereof.

9. The method of any one of claims 1-8, wherein the subject has pain catastrophizing.

10. The method of any one of claims 1-9, wherein the method further comprises increasing dopamine levels in the subject in need thereof.

11. The method of any one of claims 1-10, wherein the subject has persistent postsurgical pain (PPSP).

12. The method of claim 7, wherein the administering at least partially blocks the neuropathic pain.

13. The method of claim 7, wherein the administering enhances opioid receptor analgesia.

14. The method of claim 7, wherein the administering results in the repair of neurons.

15. The method of any one of claims 1-14, wherein the administering is daily, weekly, or monthly.

16. The method of any one of claims 1-15, wherein administering is once, twice, three, or four times per day.

17. The method of any one of claims 1-16, wherein the pharmaceutical composition is administered for about: one day, two days, three days, four days, five days, six days, one week, two weeks, three weeks, four weeks, five weeks, one month, two months, three months, four months, five months, six months, seven months, eight months, nine months, ten months, eleven months, one year, two years, or for life.

18. The method of any one of claims 1-17, wherein pharmaceutical composition comprises the polypeptide, the derivative thereof, or the salt thereof in an amount of from about 0.005 mg to about 1000 mg.

19. The method of any one of claims 1-18, wherein the pharmaceutical composition is administered by: an oral route, an injection route, a sublingual route, a buccal route, a rectal route, a vaginal route, an ocular route, an otic route, a nasal route, an internasal route, an inhalation route, a cutaneous route, a subcutaneous route, an intramuscular route, an intravenous route, a systemic route, a local route, a transdermal route, or any combination thereof.

20. The method of any one of claims 1-19, wherein the pharmaceutical composition is formulated for oral administration.

21. The method of claim 20, wherein the pharmaceutical composition is in a form of a pill or a liquid.

22. The method of any one of claims 1-21, wherein a second therapy is administered concurrently or consecutively.

23. The method of claim 22, wherein the second therapy comprises an gabapentinoid, an opioid, a voltage-gated sodium channel inhibitor, an anti-nerve growth factor, an nonsteroidal anti-inflammatory drug, aspirin, a corticosteroid, acetaminophen, a muscle relaxant, an anti-anxiety drug, an antidepressant, a cox-2 inhibitor, a local anesthetic, an anticonvulsant, a cannabinoid, an NMDA receptor antagonist, an α2-adrenergic receptor agonist or any combination thereof.

24. The method of any one of claims 22-23, wherein the pharmaceutical composition further comprises the second therapy.

25. The method of any one of claims 1-24, wherein the subject was diagnosed with pain prior to the administration.

26. The method of claim 25, wherein the diagnoses comprise an in vitro test, a physical exam, an imaging diagnostic or a combination thereof.

27. The method of any one of claims 1-26, wherein the subject is a mammal.

28. The method of claim 27, wherein the mammal is a human.

29. A method of administering a polypeptide to treat Persistent Postsurgical Pain (PPSP) in a subject in need thereof, the method comprising administering to the subject a therapeutically effective amount of a pharmaceutical composition to treat PPSP, wherein the pharmaceutical composition comprises:
a polypeptide, a derivative thereof, or a salt thereof, wherein the polypeptide comprises at least five contiguous amino acids or derivatives thereof comprising the general formula: E-F-G-H-I, wherein:
E is D-Ser, D-Thr, D-Asn, D-Glu, D-Arg, D-Ile, or D-Leu, or a derivative of any of these;
F is D-Ser, D-Thr, D-Asp, or D-Asn, or a derivative of any of these;
G is D-Thr, D-Ser, D-Asn, D-Arg, D-Gln, D-Lys, or D-Trp, or a derivative of any of these;
H is D-Tyr, or a derivative thereof; and
I is D-Thr, D-Ser, D-Arg, or Gly, or a derivative of any of these.

30. The method of claim 29, wherein the polypeptide, the derivative thereof, or the salt thereof comprises at least eight contiguous amino acids or derivatives thereof, comprising the general formula A-B-C-E-F-G-H-I, and wherein:
A is D-Ala, or a derivative thereof;
B is D-Ser, or D-Thr, or a derivative of any of these;
C is D-Ser, or D-Thr, or a derivative of any of these;
E is D-Ser, D-Thr, D-Asn, D-Glu, D-Arg, D-Ile, or D-Leu, or a derivative of any of these;
F is D-Ser, D-Thr, D-Asp, or D-Asn, or a derivative of any of these;
G is D-Thr, D-Ser, D-Asn, D-Arg, D-Gln, D-Lys, or D-Trp, or a derivative of any of these;
H is D-Tyr, or a derivative thereof; and
I is D-Thr, D-Ser, D-Arg, or Gly or a derivative of any of these.

31. The method of claim 29 or claim 30, wherein the polypeptide or the salt thereof is D-Thr, D-Thr, D-Asn, D-Tyr, and D-Thr or a salt thereof.

32. The method of any one of claims 29-31, comprising the derivative of I, wherein I is esterified, glycosylated, or amidated at the C terminus.

33. The method of any one of claims 29-32, wherein the pharmaceutical composition is in unit dose form.

34. The method of any one of claims 29-33, wherein the pharmaceutical composition further comprises a pharmaceutically acceptable: excipient, diluent, carrier, or any combination thereof.

35. The method of any one of claims 29-34, wherein the pharmaceutical composition is formulated for oral administration.

36. The method of claim 35, wherein the pharmaceutical composition is in a form of a pill or a liquid.


SEQUENCE LISTING (3 sequences)

SEQ ID NO: 1 - protein, 8 aa, Artificial Sequence
  Description of Artificial Sequence Synthetic peptide
ASTTTNYT

SEQ ID NO: 2 - protein, 5 aa, Artificial Sequence
  Description of Artificial Sequence Synthetic peptide
TTNYT

SEQ ID NO: 3 - protein, 8 aa, Artificial Sequence
  Description of Artificial Sequence Synthetic peptide
  C-term NH2
ASTTTNYT